SEQUENCE LISTING (108 sequences)

SEQ ID NO: 1 - DNA, 231 bp, Artificial Sequence
  Description of Artificial Sequence Synthetic General formula
  modified_base (1)..(231): a, c, t, g, unknown or other
  misc_feature (1)..(10): This region may encompass 0-10 nucleotides
  misc_feature (11)..(14): This region encompasses 4 nucleotides and at least the last 3 nucleotides are reverse-complementary to the first 3 nucleotides of N17 (positions 218-221)
  misc_feature (15)..(22): This region may encompass 5-8 nucleotides and is not reverse-complementary to N16 (positions 210-217)
  misc_feature (23)..(26): This region may encompass 2-4 nucleotides
  misc_feature (27)..(30): This region may encompass 2-4 nucleotides
  misc_feature (31)..(34): This region may encompass 2-4 nucleotides
  misc_feature (35)..(64): This region may encompass 3-30 nucleotides
  misc_feature (65)..(164): This region may encompass 3-100 nucleotides
  misc_feature (165)..(194): This region may encompass 3-30 nucleotides
  misc_feature (195)..(195): May or may not be present
  misc_feature (196)..(199): This region may encompass 2-4 nucleotides
  misc_feature (200)..(200): May or may not be present
  misc_feature (201)..(204): This region may encompass 2-4 nucleotides
  misc_feature (205)..(205): May or may not be present
  misc_feature (206)..(209): This region may encompass 2-4 nucleotides
  misc_feature (210)..(217): This region may encompass 5-8 nucleotides and is not reverse-complementary to N3 (positions 15-22)
  misc_feature (218)..(221): This region encompasses 4 nucleotides and at least the last 3 nucleotides are reverse-complementary to the first 3 nucleotides of N2 (positions 11-14)
  misc_feature (222)..(231): This region may encompass 0-10 nucleotides
  See specification as filed for detailed description of substitutions and preferred embodiments
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 2 - DNA, 66 bp, Artificial
  attCaadA7
cggttataacaattcattcaagccgacgccgcttcgcggcgcggcttaattcaagcgttataaccg

SEQ ID NO: 3 - DNA, 73 bp, Artificial
  attCr0
gaattcattataacggaggttacccatggattcgagttcctcgaaccatggtaaagagtgttataatggatcc

SEQ ID NO: 4 - DNA, 73 bp, Artificial
  attCr1
gaattccgtctaactcatcgcgcgtgaataaacctcttggaggttattcaccgcaaaatgttagacgggatcc

SEQ ID NO: 5 - DNA, 73 bp, Artificial
  attCr2
gaattcagggtaacgctacgcgcagtgccaagcatctatgatgctggcactcgcctcttgttaccctggatcc

SEQ ID NO: 6 - DNA, 73 bp, Artificial
  attCr3
gaattctctctaacctgctactctatagtacagtaaggtttactgactataagtcgggtgttagagaggatcc

SEQ ID NO: 7 - DNA, 73 bp, Artificial
  attCr4
gaattctttctaactccgccaacccggagaagcgtggcccacgctctccggttgatattgttagaaaggatcc

SEQ ID NO: 8 - DNA, 73 bp, Artificial
  attCr5
gaattcgccttaacaatacaggctatgttatttggtcggaccaaaaacatacctagtaggttaaggcggatcc

SEQ ID NO: 9 - DNA, 73 bp, Artificial
  attCr6
gaattcatcttaactgcttacacccgggcaacctttcctaaaggtgcccggtgtggcttgttaagatggatcc

SEQ ID NO: 10 - DNA, 75 bp, Artificial
  attCr7
gaattctcggctaaccgctcagactatcgcactacctggtttctaggcgatatcttcggagttagccgaggatcc

SEQ ID NO: 11 - DNA, 75 bp, Artificial
  attCr8
gaattctaggctaacagaacggtcaatatgagggaggctggatccccatattacctcggtgttagcctaggatcc

SEQ ID NO: 12 - DNA, 76 bp, Artificial
  attCr9
gaattcacgacgaactcagacgacagatataacctaaaagttcggtatatcttcgggccgtgttcgtcgtggatcc

SEQ ID NO: 13 - DNA, 76 bp, Artificial
  attCr10
gaattcctgcctaacgtcgtctgcagcgtcacactgtacgcatgtggacgctcagtcaaatgttaggcagggatcc

SEQ ID NO: 14 - DNA, 75 bp, Artificial
  attCr11
gaattcttacacaacggcccatactgaatcagaaatccaaacattcgattcatattcgacgttgtgtaaggatcc

SEQ ID NO: 15 - DNA, 75 bp, Artificial
  attCr12
gaattcatggctaactagtaatactcagggaatcgatcacggtgatccctgatatgctctgttagccatggatcc

SEQ ID NO: 16 - DNA, 76 bp, Artificial
  attCr13
gaattcgccactaacagtttctacgatttgaatgtcgatatcgcatcaaatctagcggctcgttagtggcggatcc

SEQ ID NO: 17 - DNA, 153 bp, Artificial
  Target sequence module 1
ctgcacgcatcggagcggtcgaaggagatcgactggtcatccggtgcgatcagcctgctcgacgagccggagccgtggcccgccggcgcgcgaccgcgccgggcgggggtctcgtcgttcggcatcagcggcaccaacgcgcacgccatcatc

SEQ ID NO: 18 - DNA, 153 bp, Artificial
  Target sequence module 1-I
ctgcacgcatcggagcggtcgaaggagataacgtggtcatctggtgcacttagcctgctcgacgagccggagccgtggcccgccggcgcgcgaccgcgccgggcgggggtctcgtcgttcggcatcaaaggcaccaatgctcatgttatcttc

SEQ ID NO: 19 - DNA, 26 bp, Artificial
  N1-N2-N3-N4-N5-N6-N7 module 1-I
agataacgtggtcatctggtgcactt

SEQ ID NO: 20 - DNA, 76 bp, Artificial
  N8 module 1-I
agcctgctcgacgagccggagccgtggcccgccggcgcgcgaccgcgccgggcgggggtctcgtcgttcggcatca

SEQ ID NO: 21 - DNA, 24 bp, Artificial
  N9-N10-N11-N13-N14-N15-N16-N17-N18 module 1-I
aaggcaccaatgctcatgttatct

SEQ ID NO: 22 - DNA, 153 bp, Artificial
  Target sequence module 1-II
ctgcacgcatcggagcggtcgaaggctataacttggtcatctggtgcacttagcctgctcgacgagccggagccgtggcccgccggcgcgcgaccgcgccgggcgggggtctcgtcgttcggcatcaaaggcaccaatgcgcatgttatagtc

SEQ ID NO: 23 - DNA, 26 bp, Artificial
  N1-N2-N3-N4-N5-N6-N7 module 1-II
ctataacttggtcatctggtgcactt

SEQ ID NO: 24 - DNA, 76 bp, Artificial; identical to SEQ ID NO: 20
  N8 module 1-II

SEQ ID NO: 25 - DNA, 24 bp, Artificial
  N9-N10-N11-N13-N14-N15-N16-N17-N18 module 1-II
aaggcaccaatgcgcatgttatag

SEQ ID NO: 26 - DNA, 153 bp, Artificial
  Target sequence module 1-III
ctgcacgcatcggagcggtcgaaggagatcgactggaataacggtgccatctcgtacacagacgagccggagccgtggcccgccggcgcgcgaccgcgccgggcgggggtctcgtcgttcggcatcactggtacgaatgcgcacgttattctc

SEQ ID NO: 27 - DNA, 26 bp, Artificial
  N1-N2-N3-N4-N5-N6-N7 module 1-III
gaataacggtgccatctcgtacacag

SEQ ID NO: 28 - DNA, 66 bp, Artificial
  N8 module 1-III
acgagccggagccgtggcccgccggcgcgcgaccgcgccgggcgggggtctcgtcgttcggcatca

SEQ ID NO: 29 - DNA, 24 bp, Artificial
  N9-N10-N11-N13-N14-N15-N16-N17-N18 module 1-III
ctggtacgaatgcgcacgttattc

SEQ ID NO: 30 - DNA, 153 bp, Artificial
  Target sequence module 2
tgccggggcgagaggtcgggcctcatcgactggtcctccggcgagatcgagctcgcagacggcgtgcgggagtggtcgcccgccgcggacggggtgcgccgggcaggtgtgtcggcgttcggggtgagcgggacgaacgcgcacgtgatcatc

SEQ ID NO: 31 - DNA, 153 bp, Artificial
  Target sequence module 2-I
tgccggggcgagaggtcgggcctgataacgtggtcgtctggttcactggagctcgcagacggcgtgcgggagtggtcgcccgccgcggacggggtgcgccgggcaggtgtgtcggcgttcggggtgacaggaaccaacgcacacgttatcatc

SEQ ID NO: 32 - DNA, 26 bp, Artificial
  N1-N2-N3-N4-N5-N6-N7 module 2-I
tgataacgtggtcgtctggttcactg

SEQ ID NO: 33 - DNA, 79 bp, Artificial
  N8 module 2-I
gagctcgcagacggcgtgcgggagtggtcgcccgccgcggacggggtgcgccgggcaggtgtgtcggcgttcggggtga

SEQ ID NO: 34 - DNA, 24 bp, Artificial
  N9-N10-N11-N13-N14-N15-N16-N17-N18 module 2-I
caggaaccaacgcacacgttatca

SEQ ID NO: 35 - DNA, 153 bp, Artificial
  Target sequence module 2-II
tgccggggcgagaggtcgggcctcatcgactggtcctccggcgagataactctggcgtctggtgcacgcgagtggtcgcccgccgcggacggggtgcgccgggcaggtgtgtcggcgttcggggtgagcggcaccaacgctcatgttatcttc

SEQ ID NO: 36 - DNA, 26 bp, Artificial
  N1-N2-N3-N4-N5-N6-N7 module 2-II
agataactctggcgtctggtgcacgc

SEQ ID NO: 37 - DNA, 58 bp, Artificial
  N8 module 2-II
gagtggtcgcccgccgcggacggggtgcgccgggcaggtgtgtcggcgttcggggtga

SEQ ID NO: 38 - DNA, 24 bp, Artificial
  N9-N10-N11-N13-N14-N15-N16-N17-N18 module 2-II
gcggcaccaacgctcatgttatct

SEQ ID NO: 39 - DNA, 153 bp, Artificial
  Target sequence module 2-III
tgccggggcgagaggtcgggcctcatcgactggtcctccggcgagatcgagctcgcagacggcgtgcgggagtggtcgcccgccgcggacgggataacacgcagttcagtaccagctttcggggtgagcggtactaacgcgcatgttatcctc

SEQ ID NO: 40 - DNA, 26 bp, Artificial
  N1-N2-N3-N4-N5-N6-N7 module 2-III
ggataacacgcagttcagtaccagct

SEQ ID NO: 41 - DNA, 25 bp, Artificial
  N9-N10-N11-N13-N14-N15-N16-N17-N18 module 2-III
agcggtactaacgcgcatgttatcc

SEQ ID NO: 42 - DNA, 153 bp, Artificial
  Target sequence module 3
ctgcacgtcgaggagcccacgccgcacgtcgactggtcgtccggcggcgtggcgctgctggcgggcaaccagccgtggcggcgcggcgagcggactcggcgcgccgctgtttccgcgttcgggatcagcgggacgaatgcgcacgtgatcgtc

SEQ ID NO: 43 - DNA, 153 bp, Artificial
  Target sequence module 3-I
ctgcacgtcgaggagcccacgccgcacgtcgactggtcgtccggcggcgtggcgctgctggcgggcaaccagccgtggcggcgcggcgagcgtacaacgaggagttctgtaccagctttcgggatcagcggtacaaacgcgcacgttgtactc

SEQ ID NO: 44 - DNA, 26 bp, Artificial
  N1-N2-N3-N4-N5-N6-N7 module 3-I
gtacaacgaggagttctgtaccagct

SEQ ID NO: 45 - DNA, 25 bp, Artificial
  N9-N10-N11-N13-N14-N15-N16-N17-N18 module 3-I
agcggtacaaacgcgcacgttgtac

SEQ ID NO: 46 - DNA, 153 bp, Artificial
  Target sequence module 3-II
ctgcacgtcgaggagcccacgccgcacgtcgactggtcgtccggcggcgtggcgctgctggcgggcaaccagccgtggcggcgcggcgagcgtacaacgcgggctgcagtaccagctttcgggatcagcggtactcaggctcacgttgtactc

SEQ ID NO: 47 - DNA, 26 bp, Artificial
  N1-N2-N3-N4-N5-N6-N7 module 3-II
gtacaacgcgggctgcagtaccagct

SEQ ID NO: 48 - DNA, 25 bp, Artificial
  N9-N10-N11-N13-N14-N15-N16-N17-N18 module 3-II
agcggtactcaggctcacgttgtac

SEQ ID NO: 49 - DNA, 153 bp, Artificial
  Target sequence module 3-III
ctgcacgtcgaggagcccacgccgcacgtcgactggtcgtccggcggcgtggcgctgctggcgggcaaccagccgtggcggcgcggcgagcgacaacgaagacgtctggtgcacgcgttcgggatcagcggcaccaacgctcacgttgtcgtc

SEQ ID NO: 50 - DNA, 26 bp, Artificial
  N1-N2-N3-N4-N5-N6-N7 module 3-III
cgacaacgaagacgtctggtgcacgc

SEQ ID NO: 51 - DNA, 11 bp, Artificial
  N8 module 3-III
gttcgggatca

SEQ ID NO: 52 - DNA, 24 bp, Artificial
  N9-N10-N11-N13-N14-N15-N16-N17-N18 module 3-III
gcggcaccaacgctcacgttgtcg

SEQ ID NO: 53 - DNA, 153 bp, Artificial
  Target sequence module 4
ttgcacgccgacgagctgtccccgcacatcgactgggagtcgggggccgtggaggtgctgcgcgaggaggtgccgtggccggcgggtgagcgcccccggcgggcgggggtgtcgtccttcggcgtcagcggaaccaacgcgcacgtgatcgtc

SEQ ID NO: 54 - DNA, 153 bp, Artificial
  Target sequence module 4-I
ttgcacgccgacgagctgtccccgcacatcgactgggagtcgggacaactggaagttctgcgccaggaggtgccgtggccggcgggtgagcgcccccggcgggcgggggtgtcgtccttcggcgtctccggcgcaaactcacatgttgtcctc

SEQ ID NO: 55 - DNA, 26 bp, Artificial
  N1-N2-N3-N4-N5-N6-N7 module 4-I
ggacaactggaagttctgcgccagga

SEQ ID NO: 56 - DNA, 58 bp, Artificial
  N8 module 4-I
ggtgccgtggccggcgggtgagcgcccccggcgggcgggggtgtcgtccttcggcgtc

SEQ ID NO: 57 - DNA, 25 bp, Artificial
  N9-N10-N11-N13-N14-N15-N16-N17-N18 module 4-I
tccggcgcaaactcacatgttgtcc

SEQ ID NO: 58 - DNA, 153 bp, Artificial
  Target sequence module 4-II
ttgcacgccgacgagctgtccccgcacatcgactgggagtcgggggccgtggaggtgctgcgcgaggaggtgccgtggccggcgggtgagcgcccccacaacgcaggcgtctcgtccaccggcgtcacggggacgaacgctcacgttgtggtc

SEQ ID NO: 59 - DNA, 26 bp, Artificial
  N1-N2-N3-N4-N5-N6-N7 module 4-II
ccacaacgcaggcgtctcgtccaccg

SEQ ID NO: 60 - DNA, 24 bp, Artificial
  N9-N10-N11-N13-N14-N15-N16-N17-N18 module 4-II
cggggacgaacgctcacgttgtgg

SEQ ID NO: 61 - DNA, 153 bp, Artificial
  Target sequence module 4-III
ttgcacgccgacgagctgtccccgcacatcgactgggagtcgggggccgtggaggtgctgcgcgaggaggtgccgtggccggcgggtgagcgccctaaccgcgcaggcgtttccagcttcggcgtcagcggaaaccctgcgcatgttagggtc

SEQ ID NO: 62 - DNA, 26 bp, Artificial
  N1-N2-N3-N4-N5-N6-N7 module 4-III
ccctaaccgcgcaggcgtttccagct

SEQ ID NO: 63 - DNA, 25 bp, Artificial
  N9-N10-N11-N13-N14-N15-N16-N17-N18 module 4-III
agcggaaaccctgcgcatgttaggg

SEQ ID NO: 64 - DNA, 153 bp, Artificial
  Target sequence module 5
ctgcacttcgacgagccctcgccgcagatcgagtgggacctgggcgcggtgtcggtggtgtcgcaggcgcggtcgtggcccgccggcgagaggccccgcagggcgggcgtctcctcgttcggcatcagcggcaccaacgcgcacgtcatcgtc

SEQ ID NO: 65 - DNA, 153 bp, Artificial
  Target sequence module 5-I
ctgcacttcgacgagccctcgccgcagatcgagtgggacctgggcgcgataacagttgcgtctcgtgcacgctcgtggcccgccggcgagaggccccgcagggcgggcgtctcctcgttcggcatcagcggcacgaacgctcatgttatcgtc

SEQ ID NO: 66 - DNA, 26 bp, Artificial
  N1-N2-N3-N4-N5-N6-N7 module 5-I
cgataacagttgcgtctcgtgcacgc

SEQ ID NO: 67 - DNA, 55 bp, Artificial
  N8 module 5-I
tcgtggcccgccggcgagaggccccgcagggcgggcgtctcctcgttcggcatca

SEQ ID NO: 68 - DNA, 24 bp, Artificial
  N9-N10-N11-N13-N14-N15-N16-N17-N18 module 5-I
gcggcacgaacgctcatgttatcg

SEQ ID NO: 69 - DNA, 153 bp, Artificial
  Target sequence module 5-II
ctgcacttcgacgagccctcgccgcagatcgagtgggacctgggcgcgataactgtagtgtctcgtgcacgctcgtggcccgccggcgagaggccccgcagggcgggcgtctcctcgttcggcatcagcggcacgaacactcatgttatcgtc

SEQ ID NO: 70 - DNA, 26 bp, Artificial
  N1-N2-N3-N4-N5-N6-N7 module 5-II
cgataactgtagtgtctcgtgcacgc

SEQ ID NO: 71 - DNA, 55 bp, Artificial; identical to SEQ ID NO: 67
  N8 module 5-II

SEQ ID NO: 72 - DNA, 24 bp, Artificial
  N9-N10-N11-N13-N14-N15-N16-N17-N18 module 5-II
gcggcacgaacactcatgttatcg

SEQ ID NO: 73 - DNA, 153 bp, Artificial
  Target sequence module 5-III
ctgcacttcgacgagccctcgccgcagatcgagtgggacctgggcgcgataacggtagtgtctcgtgcacgctcgtggcccgccggcgagaggccccgcagggcgggcgtctcctcgttcggcatcagcggcacgaacattcatgttatcgtc

SEQ ID NO: 74 - DNA, 26 bp, Artificial
  N1-N2-N3-N4-N5-N6-N7 module 5-III
cgataacggtagtgtctcgtgcacgc

SEQ ID NO: 75 - DNA, 55 bp, Artificial; identical to SEQ ID NO: 67
  N8 module 5-III

SEQ ID NO: 76 - DNA, 24 bp, Artificial
  N9-N10-N11-N13-N14-N15-N16-N17-N18 module 5-III
gcggcacgaacattcatgttatcg

SEQ ID NO: 77 - DNA, 153 bp, Artificial
  Target sequence module 6
tgccgcggcgagcggtcgccgctgatcgaatggtcctcgggtggtgtggaacttgccgaggccgtgagcccgtggcctccggccgcggacggggtgcgccgggccggtgtgtcggcgttcggggtgagcgggacgaacgcgcacgtgatcatc

SEQ ID NO: 78 - DNA, 153 bp, Artificial
  Target sequence module 6-I
tgccgcggagaacgtagtcctctcgtgcactggtcctcgggtggtgtggaacttgccgaggccgtgagcccgtggcctccggccgcggacggggtgcgccgggccggtgtgtcggcgttcggggtgacaggcacgaaggcacacgttctcctc

SEQ ID NO: 79 - DNA, 26 bp, Artificial
  N1-N2-N3-N4-N5-N6-N7 module 6-I
ggagaacgtagtcctctcgtgcactg

SEQ ID NO: 80 - DNA, 95 bp, Artificial
  N8 module 6-I
gtcctcgggtggtgtggaacttgccgaggccgtgagcccgtggcctccggccgcggacggggtgcgccgggccggtgtgtcggcgttcggggtga

SEQ ID NO: 81 - DNA, 24 bp, Artificial
  N9-N10-N11-N13-N14-N15-N16-N17-N18 module 6-I
caggcacgaaggcacacgttctcc

SEQ ID NO: 82 - DNA, 153 bp, Artificial
  Target sequence module 6-II
tgccgcggcgagcggtcgccgctgataacttggtcatctggtggagtggaacttgccgaggccgtgagcccgtggcctccggccgcggacggggtgcgccgggccggtgtgtcggcgttcggggtgacacccaccaatgctcatgttatcatc

SEQ ID NO: 83 - DNA, 26 bp, Artificial
  N1-N2-N3-N4-N5-N6-N7 module 6-II
tgataacttggtcatctggtggagtg

SEQ ID NO: 84 - DNA, 79 bp, Artificial
  N8 module 6-II
gaacttgccgaggccgtgagcccgtggcctccggccgcggacggggtgcgccgggccggtgtgtcggcgttcggggtga

SEQ ID NO: 85 - DNA, 24 bp, Artificial
  N9-N10-N11-N13-N14-N15-N16-N17-N18 module 6-II
cacccaccaatgctcatgttatca

SEQ ID NO: 86 - DNA, 153 bp, Artificial
  Target sequence module 6-III
tgccgcggcgagcggtcgccgctgatcgaatgggataacggtggcgtctcgtgcacgcaggccgtgagcccgtggcctccggccgcggacggggtgcgccgggccggtgtgtcggcgttcggggtgagcggcacgaacgcccatgttatcctc

SEQ ID NO: 87 - DNA, 26 bp, Artificial
  N1-N2-N3-N4-N5-N6-N7 module 6-III
ggataacggtggcgtctcgtgcacgc

SEQ ID NO: 88 - DNA, 69 bp, Artificial
  N8 module 6-III
aggccgtgagcccgtggcctccggccgcggacggggtgcgccgggccggtgtgtcggcgttcggggtga

SEQ ID NO: 89 - DNA, 24 bp, Artificial
  N9-N10-N11-N13-N14-N15-N16-N17-N18 module 6-III
gcggcacgaacgcccatgttatcc

SEQ ID NO: 90 - DNA, 126 bp, Artificial
  attCr module 1-I
agataacgtggtcatctggtgcacttagcctgctcgacgagccggagccgtggcccgccggcgcgcgaccgcgccgggcgggggtctcgtcgttcggcatcaaaggcaccaatgctcatgttatct

SEQ ID NO: 91 - DNA, 126 bp, Artificial
  attCr module 1-II
ctataacttggtcatctggtgcacttagcctgctcgacgagccggagccgtggcccgccggcgcgcgaccgcgccgggcgggggtctcgtcgttcggcatcaaaggcaccaatgcgcatgttatag

SEQ ID NO: 92 - DNA, 116 bp, Artificial
  attCr module 1-III
gaataacggtgccatctcgtacacagacgagccggagccgtggcccgccggcgcgcgaccgcgccgggcgggggtctcgtcgttcggcatcactggtacgaatgcgcacgttattc

SEQ ID NO: 93 - DNA, 129 bp, Artificial
  attCr module 2-I
tgataacgtggtcgtctggttcactggagctcgcagacggcgtgcgggagtggtcgcccgccgcggacggggtgcgccgggcaggtgtgtcggcgttcggggtgacaggaaccaacgcacacgttatca

SEQ ID NO: 94 - DNA, 108 bp, Artificial
  attCr module 2-II
agataactctggcgtctggtgcacgcgagtggtcgcccgccgcggacggggtgcgccgggcaggtgtgtcggcgttcggggtgagcggcaccaacgctcatgttatct

SEQ ID NO: 95 - DNA, 60 bp, Artificial
  attCr module 2-III
ggataacacgcagttcagtaccagctttcggggtgagcggtactaacgcgcatgttatcc

SEQ ID NO: 96 - DNA, 60 bp, Artificial
  attCr module 3-I
gtacaacgaggagttctgtaccagctttcgggatcagcggtacaaacgcgcacgttgtac

SEQ ID NO: 97 - DNA, 60 bp, Artificial
  attCr module 3-II
gtacaacgcgggctgcagtaccagctttcgggatcagcggtactcaggctcacgttgtac

SEQ ID NO: 98 - DNA, 61 bp, Artificial
  attCr module 3-III
cgacaacgaagacgtctggtgcacgcgttcgggatcagcggcaccaacgctcacgttgtcg

SEQ ID NO: 99 - DNA, 109 bp, Artificial
  attCr module 4-I
ggacaactggaagttctgcgccaggaggtgccgtggccggcgggtgagcgcccccggcgggcgggggtgtcgtccttcggcgtctccggcgcaaactcacatgttgtcc

SEQ ID NO: 100 - DNA, 56 bp, Artificial
  attCr module 4-II
ccacaacgcaggcgtctcgtccaccggcgtcacggggacgaacgctcacgttgtgg

SEQ ID NO: 101 - DNA, 59 bp, Artificial
  attCr module 4-III
ccctaaccgcgcaggcgtttccagcttcggcgtcagcggaaaccctgcgcatgttaggg

SEQ ID NO: 102 - DNA, 105 bp, Artificial
  attCr module 5-I
cgataacagttgcgtctcgtgcacgctcgtggcccgccggcgagaggccccgcagggcgggcgtctcctcgttcggcatcagcggcacgaacgctcatgttatcg

SEQ ID NO: 103 - DNA, 105 bp, Artificial
  attCr module 5-II
cgataactgtagtgtctcgtgcacgctcgtggcccgccggcgagaggccccgcagggcgggcgtctcctcgttcggcatcagcggcacgaacactcatgttatcg

SEQ ID NO: 104 - DNA, 105 bp, Artificial
  attCr module 5-III
cgataacggtagtgtctcgtgcacgctcgtggcccgccggcgagaggccccgcagggcgggcgtctcctcgttcggcatcagcggcacgaacattcatgttatcg

SEQ ID NO: 105 - DNA, 145 bp, Artificial
  attCr module 6-I
ggagaacgtagtcctctcgtgcactggtcctcgggtggtgtggaacttgccgaggccgtgagcccgtggcctccggccgcggacggggtgcgccgggccggtgtgtcggcgttcggggtgacaggcacgaaggcacacgttctcc

SEQ ID NO: 106 - DNA, 105 bp, Artificial
  attCr module 6-II
tgataacttggtcatctggtggagtggaacttgccgaggccgtgagcccgtggcctccggccgcggacggggtgcgccgggccggtgtgtcggcgttcggggtga

SEQ ID NO: 107 - DNA, 119 bp, Artificial
  attCr module 6-III
ggataacggtggcgtctcgtgcacgcaggccgtgagcccgtggcctccggccgcggacggggtgcgccgggccggtgtgtcggcgttcggggtgagcggcacgaacgcccatgttatcc

SEQ ID NO: 108 - DNA, 96 bp, Artificial
  bottom strand of attCaadA7
ggatccgtctaacgcttgaattaagccgcgccgcgaagcggcgtcggcttgaatgaattgttagacgaattccacccaccaatgctcatgttatca

(19) United States
(12) Patent Application Publication
(10) Pub. No.: US 2020/0165600 A1
(43) Pub. Date: May 28, 2020

(54) SYNTHETIC ATTC RECOMBINATION SITES FOR PROTEIN DOMAIN SHUFFLING

(71) Applicants: INSTITUT PASTEUR, Paris (FR); UNIVERSITE PARIS DESCARTES, Paris (FR)

(72) Inventors: Didier MAZEL, Suresnes (FR); Aleksandra NIVINA, Riga (LV); David BIKARD, Paris (FR); Maj Svea GRIEB, Gottingen (DE)

(21) Appl. No.: 16/316,476

(22) PCT Filed: Jul. 28, 2017
(86) PCT No.: PCT/EP2017/069156

§ 371 Date: Jan. 9, 2019

Related U.S. Application Data
(60) Provisional application No. 62/368,201, filed on Jul. 29, 2016.

(51) Int. Cl. C12N 15/10

(52) U.S. Cl. CPC C12N 15/1027; C12N 15/1082

(57) ABSTRACT

Recombinant nucleic acids comprising a protein coding sequence of a multidomain protein, wherein the protein coding sequence comprises at least one synthetic attC recombination site are provided. Cells comprising the recombinant nucleic acids and methods of making the recombinant nucleic acids are also provided, among other things.

The instant application contains a Sequence Listing which has been filed electronically in ASCII format and is hereby incorporated by reference in its entirety. Said ASCII copy, created on Sep. 18, 2019, is named DI2016-16A_SL.txt and is 30,253 bytes in size.

The present disclosure is related to the fields of synthetic biology, genetic engineering, molecular biology, microbiology and recombineering. In particular, the present disclosure relates to the design, testing and use of polynucleotides coding for synthetic integron recombination sites for protein domain shuffling and other related applications. The present disclosure also relates to vectors that encode recombinant modular type I polyketide synthases (PKS) and recombinant non-ribosomal polypeptide synthases (NRPS), the vectors comprising at least one synthetic integron recombination site. The present disclosure also relates to combinatorial libraries of recombinant PKS and/or recombinant NRPS, among other things.

Site-specific DNA recombination is one of the basic tools in bacterial genetic engineering. It allows combining heterologous sequences in expression vectors, integrating synthetic constructions into the genome of host organisms, manipulating large DNA fragments in vivo and much more. Contrary to CRISPR-Cas systems that are mostly used in eukaryotic organisms and have limited efficiency for bacterial genome editing, a large number of site-specific recombination systems are highly efficient in prokaryotes. However, most recombination systems require recombination sites with a predefined sequence that cannot be easily modified, or even has to be kept constant. Such requirements limit the possibilities to insert these recombination sites into DNA regions that already carry a function, such as protein coding sequences, promoters etc. This makes it close to impossible to design a system for site-specific recombination within these DNA regions, for instance for protein domain recombination.

There is a need in the art for a system that allows recombination between protein domains within proteins while maintaining the open reading from and biological function of the encoded protein. This invention meets this and other needs.

### SUMMARY

In a first aspect, this invention provides recombinant nucleic acids comprising a protein coding sequence of a multidomain protein, wherein the protein coding sequence comprises at least one synthetic attC recombination site. In some embodiments the recombinant nucleic acid is a DNA. In some embodiments the recombinant nucleic acid is isolated. In some embodiments the recombinant nucleic acid is a vector. In some embodiments the recombinant nucleic acid is a chromosome. In some embodiments the at least one synthetic attC recombination site has a size of 60 nucleotides or more. In some embodiments the multi-domain protein is a modular type I polyketide synthase (PKS). In some embodiments the multi-domain protein is a non-ribosomal polypeptide synthase (NRPS). In some embodiments the recombinant nucleic acid comprises at least one recombined synthetic attC recombination site.

In another aspect, this invention provides recombinant cells comprising at least on nucleic acid comprising a protein coding sequence of a multidomain protein, wherein the protein coding sequence comprises at least one synthetic attC recombination site. In some embodiments the recombinant nucleic acid is a DNA. In some embodiments the recombinant nucleic acid is a vector. In some embodiments the recombinant nucleic acid is a chromosome. In some embodiments the at least one synthetic attC recombination site has a size of 60 nucleotides or more. In some embodiments the multi-domain protein is a modular type I polyketide synthase (PKS). In some embodiments the multi-domain protein is a non-ribosomal polypeptide synthase (NRPS). In some embodiments the recombinant nucleic acid comprises at least one recombined synthetic attC recombination site.

In another aspect, this invention provides a library comprising a plurality of different recombinant nucleic acids comprising a protein coding sequence of a multidomain protein, wherein the protein coding sequence comprises at least one synthetic attC recombination site. In some embodiments the recombinant nucleic acid is a DNA. In some embodiments the recombinant nucleic acid is a vector. In some embodiments the recombinant nucleic acid is a chromosome. In some embodiments the at least one synthetic attC recombination site has a size of 60 nucleotides or more. In some embodiments the multi-domain protein is a modular type I polyketide synthase (PKS). In some embodiments the multi-domain protein is a non-ribosomal polypeptide synthase (NRPS). In some embodiments the recombinant nucleic acid comprises at least one recombined synthetic attC recombination site.

In another aspect, this invention provides a library comprising a plurality of different recombinant cells comprising at least on nucleic acid comprising a protein coding sequence of a multidomain protein, wherein the protein coding sequence comprises at least one synthetic attC recombination site. In some embodiments the recombinant nucleic acid is a DNA. In some embodiments the recombinant nucleic acid is a vector. In some embodiments the recombinant nucleic acid is a chromosome. In some embodiments the at least one synthetic attC recombination site has a size of 60 nucleotides or more. In some embodiments the multi-domain protein is a modular type I polyketide synthase (PKS). In some embodiments the multi-domain protein is a non-ribosomal polypeptide synthase (NRPS). In some embodiments the recombinant nucleic acid comprises at least one recombined synthetic attC recombination site.

In another aspect, this invention provides methods of making a recombinant nucleic acid encoding a recombinant multidomain protein, comprising: providing a first recombinant nucleic acid comprising a protein coding sequence of a multidomain protein, wherein the protein coding sequence comprises at least one synthetic attC recombination site; providing a second recombinant nucleic acid comprising a protein coding sequence of a multidomain protein, wherein the protein coding sequence comprises at least one synthetic attC recombination site; and contacting the first and second recombinant nucleic acids with an integrase protein to thereby induce recombination between the at least one synthetic attC recombination site present in the first recombinant nucleic acid and the at least one synthetic attC recombination site present in the second recombinant nucleic acid, to thereby provide a recombined recombinant nucleic acid that encodes the recombinant multidomain protein. In some embodiments the first and second recombinant nucleic acids are present on different chromosomes and/or vectors. In some embodiments the first and second recombinant nucleic acids are present on a single chromosome or vector. In some embodiments, contacting the recombinant nucleic acid(s) with integrase protein is by a process comprising introducing a recombinant nucleic acid encoding the integrase protein into a cell comprising the recombinant nucleic acid(s) and expressing the integrase protein. In some embodiments, the recombinant multidomain protein is a recombinant PKS. In some embodiments, the recombinant multidomain protein is a recombinant NRPS.

In another aspect, this invention provides methods of making a recombinant nucleic acid encoding a recombinant multidomain protein, comprising: providing a recombinant nucleic acid comprising a plurality of protein coding sequence of multidomain proteins, wherein each of the protein coding sequences comprises a synthetic attC recombination site; and contacting the recombinant nucleic acid with an integrase protein to thereby induce recombination between at least one pair of the synthetic attC recombination sites, to thereby provide a recombined recombinant nucleic acid that encodes the recombinant multidomain protein. In some embodiments the recombinant nucleic acid is a vector. In some embodiments the recombinant nucleic acid is a chromosome. In some embodiments, contacting the recombinant nucleic acid(s) with integrase protein is by a process comprising introducing a recombinant nucleic acid encoding the integrase protein into a cell comprising the recombinant nucleic acid(s) and expressing the integrase protein. In some embodiments, the recombinant multidomain protein is a recombinant PKS. In some embodiments, the recombinant multidomain protein is a recombinant NRPS.

In another aspect, this invention provides methods of making a recombinant multidomain protein, comprising making a recombinant nucleic acid encoding a multidomain protein by a method of the invention and expressing the recombinant multidomain protein.

In another aspect, this invention provides a recombinant cell comprising a recombinant nucleic acid made by the a method of the invention. In some embodiments the recombinant nucleic acid is a recombinant vector. In some embodiments the recombinant nucleic acid is a recombinant chromosome.

In another aspect, this invention provides a library comprising a plurality of different recombinant nucleic acids of the invention. In some embodiments the nucleic acids in the library are made according to a method of the invention.

In another aspect, this invention provides a library comprising a plurality of different recombinant cells of the invention.

In another aspect, this invention provides a library comprising a plurality of different recombinant multidomain proteins of the invention.

### BRIEF DESCRIPTION OF THE DRAWINGS

FIG. 1 shows results of the EMSA performed with IntI1 integrase purified with an MBP tag, and an oligonucleotide corresponding to the bottom strand of attC_{aadA7} (sequence SEQ ID NO: 108, i.e.:

GGATCCGTCTAACGCTTGAATTAAGCCGCGCCGCGAAGCGGCGTCGGCTT GAATGAATTGTTAGACGAATTC). The appearance of shifted bands (at positions marked as “shift” 1-4) in the presence of IntI1 reflects the binding of the tested oligonucleotide by the integron integrase.

FIG. 2 shows recombination frequencies of a natural highly recombinogenic attC site attC_{aadA7} and 14 synthetic attC recombination sites (attCr0-attCr13). These results were obtained through a suicidal conjugation assay described below.

FIG. 3 shows recombination frequencies of a natural highly recombinogenic attC site attC_{aadA7} and 18 synthetic attC recombination sites (module 1-I to module 6-III). These results were obtained through the method “Evaluating the Performance of Selected Synthetic attC Recombination Sites in Recombination” described below.

### DETAILED DESCRIPTION

### A. Introduction

This disclosure provides methods for generating synthetic recombination sites with tailored sequences that can be embedded into a desired DNA region while preserving its functionality. In particular, the disclosure provides synthetic recombination sites which may be embedded within an open reading frame of a user's choice in a way that causes minimal changes to the amino acid sequence upon translation. Indeed, even though in a 50 amino acid target region approximately 20-30% of codons may be modified to accommodate the structure, the algorithm uses the redundancy of the genetic code to decrease the number of mutations on the protein level down to only, for example, 4-7 amino acids. Moreover, in some embodiments all or some of these mutations preserve the chemical characteristics of the residues. These synthetic sites are based on attC recombination sites of the integron, a bacterial recombination system that allows in vivo shuffling of DNA fragments.

The inventors have surprisingly discovered that attC sites have extremely few requirements on the primary sequence level, and identified requirements on the level of DNA secondary structure. This has allowed the inventors to design an algorithm capable of preserving these structural requirements, while tailoring the sequence toward the one defined by the user.

### B. Terminology and Definitions

Unless otherwise defined herein, scientific and technical terms used in connection with the present disclosure shall have the meanings that are commonly understood by those of ordinary skill in the art. Generally, nomenclatures used in connection with, and techniques of, biochemistry, enzymology, molecular and cellular biology, microbiology, genetics and protein and nucleic acid chemistry and hybridization described herein are those well-known and commonly used in the art. The materials, methods, and examples are illustrative only and not intended to be limiting.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3d ed., Cold Spring Harbor Laboratory Press, Cold Spring Harbor, N.Y. (2001); Ausubel et al., Current Protocols in Molecular Biology, Greene Publishing Associates (1992, and Supplements to 2002); Handbook of Biochemistry: Section A Proteins, Vol I, CRC Press (1976); Handbook of Biochemistry: Section A Proteins, Vol II, CRC Press (1976).

Before the present nucleic acids, cells, proteins, compositions, libraries methods, and other embodiments are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “comprising” as used herein is synonymous with “including” or “containing,” and is inclusive or open-ended and does not exclude additional, unrecited members, elements or method steps.

The term “multidomain protein” refers to a protein comprising a plurality of domains that each has a separate function in the synthesis of a final product from building blocks. Two exemplary multidomain proteins are modular type I polyketide synthase (PKS) and non-ribosomal polypeptide synthase (NRPS).

As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The term “peptide” as used herein refers to a short polypeptide, e.g., one that typically contains less than about 50 amino acids and more typically less than about 30 amino acids. The term as used herein encompasses analogs and mimetics that mimic structural and thus biological function.

The term “polypeptide” encompasses both naturally-occurring and non-naturally occurring proteins, and fragments, mutants, derivatives and analogs thereof. A polypeptide may be monomeric or polymeric. Further, a polypeptide may comprise a number of different domains each of which has one or more distinct activities. For the avoidance of doubt, a “polypeptide” may be any length greater two amino acids.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from a cell in which it was synthesized.

The term “fusion protein” refers to a polypeptide comprising a polypeptide or fragment coupled to heterologous amino acid sequences. Fusion proteins are useful because they can be constructed to contain two or more desired functional elements that can be from two or more different proteins. A fusion protein comprises at least 10 contiguous amino acids from a polypeptide of interest, or at least 20 or 30 amino acids, or at least 40, 50 or 60 amino acids, or at least 75, 100 or 125 amino acids. The heterologous polypeptide included within the fusion protein is usually at least 6 amino acids in length, or at least 8 amino acids in length, or at least 15, 20, or 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, a protein has “homology” or is “homologous” to a second protein if the nucleic acid sequence that encodes the protein has a similar sequence to the nucleic acid sequence that encodes the second protein. Alternatively, a protein has homology to a second protein if the two proteins have similar amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89.

The following six groups each contain amino acids that are conservative substitutions for one another: 1) Serine, Threonine; 2) Aspartic Acid, Glutamic Acid; 3) Asparagine, Glutamine; 4) Arginine, Lysine; 5) Isoleucine, Leucine, Methionine, Alanine, Valine, and 6) Phenylalanine, Tyrosine, Tryptophan.

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

An exemplary algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Exemplary parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default);

Penalty Matrix: BLOSUM62. The length of polypeptide sequences compared for homology will generally be at least about 16 amino acid residues, or at least about 20 residues, or at least about 24 residues, or at least about 28 residues, or more than about 35 residues. When searching a database containing sequences from a large number of different organisms, it may be useful to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990). For example, percent sequence identity between amino acid sequences can be determined using FASTA with its default parameters (a word size of 2 and the PAM250 scoring matrix), as provided in GCG Version 6.1, herein incorporated by reference.

In some embodiments, polymeric molecules (e.g., a polypeptide sequence or nucleic acid sequence) are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar. The term “homologous” necessarily refers to a comparison between at least two sequences (nucleotides sequences or amino acid sequences). In some embodiments, two nucleotide sequences are considered to be homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for nucleotide sequences to be considered homologous. In some embodiments of nucleotide sequences less than 60 nucleotides in length, homology is determined by the ability to encode a stretch of at least 4-5 uniquely specified amino acids. In some embodiments, two protein sequences are considered to be homologous if the proteins are at least about 50% identical, at least about 60% identical, at least about 70% identical, at least about 80% identical, or at least about 90% identical for at least one stretch of at least about 20 amino acids.

As used herein, “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

The term “polynucleotide”, “nucleic acid molecule”, “nucleic acid”, or “nucleic acid sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

A “synthetic” RNA, DNA or a mixed polymer is one created outside of a cell, for example one synthesized chemically, or one that does not occur in nature.

The term “nucleic acid fragment” as used herein refers to a nucleic acid sequence that has a deletion, e.g., a 5′-terminal or 3′-terminal deletion compared to a full-length reference nucleotide sequence. In an embodiment, the nucleic acid fragment is a contiguous sequence in which the nucleotide sequence of the fragment is identical to the corresponding positions in the naturally-occurring sequence. In some embodiments fragments are at least 10, 15, 20, or 25 nucleotides long, or at least 20, 30, 40, 50, 60, 70, 80, 90, 100, 110, 120, 130, 140, or 150 nucleotides long. In some embodiments a fragment of a nucleic acid sequence is a fragment of an open reading frame sequence. In some embodiments such a fragment encodes a polypeptide fragment (as defined herein) of the protein encoded by the open reading frame nucleotide sequence.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32, and even more typically at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, or at least about 90%, or at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tm) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

As used herein, an “expression control sequence” refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites);

sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences and fusion partner sequences.

As used herein, “operatively linked” or “operably linked” expression control sequences refers to a linkage in which the expression control sequence is contiguous with the gene of interest to control the gene of interest, as well as expression control sequences that act in trans or at a distance to control the gene of interest.

As used herein, a “vector” is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

A “recombinant vector” is a vector into which the coding sequence for a recombinant protein has been inserted.

The term “recombinant host cell” (or simply “recombinant cell” or “host cell”), as used herein, is intended to refer to a cell into which a recombinant nucleic acid such as a recombinant vector has been introduced. In some instances the word “cell” is replaced by a name specifying a type of cell. For example, a “recombinant microorganism” is a recombinant host cell that is a microorganism host cell. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell,” “recombinant cell,” and “host cell”, as used herein.

A “protein coding sequence” or “open reading frame” is a sequence of nucleotides that encodes a polypeptide or protein. The termini of the coding sequence are a start codon and a stop codon.

As used herein, a “selectable marker” is a marker that confers upon cells that possess the marker the ability to grow in the presence or absence of an agent that inhibits or stimulates, respectively, growth of similar cells that do not express the marker. Such cells can also be said to have a “selectable phenotype” by virtue of their expression of the selectable marker. For example, the ampicillin resistance gene (AmpR) confers the ability to grow in the presence of ampicillin on cells which possess and express the gene. (See Sutcliffe, J. G., Proc Natl Acad Sci USA. 1978 August; 75 (8): 3737-3741.) Other nonlimiting examples include genes that confer resistance to chloramphenicol, kanamycin, and tetracycline. Other markers include URA3, TRP and LEU, that allow growth in the absence of said uracil, tryptophan and leucine, respectively.

As used herein, a “screenable marker” is a detectable label that that can be used as a basis to identify cells that express the marker. Such cells can also be said to have a “screenable phenotype” by virtue of their expression of the screenable marker. Suitable markers include a radiolabel, a fluorescent label, a nuclear magnetic resonance active label, a luminescent label, a chromophore label, a positron emitting isotope for PET scanner, chemiluminescence label, or an enzymatic label. Fluorescent labels include but are not limited to, green fluorescent protein (GFP), fluorescein, and rhodamine. Chemiluminescence labels include but are not limited to, luciferase and β-galactosidase. Enzymatic labels include but are not limited to peroxidase and phosphatase. A histag may also be a detectable label. In some embodiments a heterologous nucleic acid is introduced into a cell and the cell then expresses a protein that is or comprises the label. For example, the introduced nucleic acid can comprise a coding sequence for GFP operatively linked to a regulatory sequence active in the cell.

As used herein, a “starting multidomain protein” is a known functional multidomain protein. In some embodiments the starting multidomain protein is a naturally occurring multidomain protein.

As used herein, a “modified derivative” of a starting multidomain protein, is a variant of a starting multidomain protein that has an amino acid sequence that differs from the amino acid sequence of the starting multidomain protein at at least one amino acid position. In some emdodiments the amino acid sequence of the modified derivative is substantially similar to the amino acid sequence of the starting multidomain protein. In some emdodiments the amino acid sequence of the modified derivative is substantially homologous to the amino acid sequence of the starting multidomain protein.

### C. Synthetic attC Recombination Sites

Integron is a bacterial site-specific DNA recombination system¹. It is composed of a stable platform and a variable cassette array. The platform contains the integron integrase gene (intI) regulated by its promoter, the integration site (attI) and the cassette promoter Pc. Cassettes are circular non-replicative elements containing a promoterless gene and a cassette recombination site attC. Upon integration into the integron platform, these cassettes form an array and the associated promoterless genes can be transcribed from the Pc promoter of the platform. This integration preferentially occurs through an attI×attC reaction, but cassettes can also be integrated through an attC×attC reaction, into one of the attC sites of the array².

While chromosomal integrons are sedentary components of bacterial genomes and contain genes playing a broad role in bacterial adaptation, mobile integrons are commonly found to be components of transposons and conjugative plasmids³. This facilitates their dissemination among Gram-negative bacterial populations and the propagation of associated antibiotic resistance genes. Among others, Class 1 integron system is historically involved in the spread of multiresistance, and has been detected in a broad range of Gram-negative bacteria^{4,5}.

The attC sites of mobile integrons have very low sequence similarity. Despite this lack of sequence conservation, integron integrase is capable of recognizing and efficiently recombining them. This is due to the unusual nature of attC recombination sites, which are recognized by the integrase as folded single-stranded DNA^{6,7}. Also, the integrase shows strand specificity for bottom strands of attC sites, recognizing and recombining them much more efficiently than top strands⁸. This strand specificity is very important for the integration of cassettes in correct orientation, so that the open reading frame encoded in the cassette can be transcribed from the promoter upstream.

The compositions and methods of this disclosure are based in part on the discovery of a set of structural features and sequence properties that allow efficient recognition and recombination of bottom strands of attC sites by the integrase.^{9,18}

The structure of the folded single-stranded attC site is an imperfect hairpin, which is also the form that is bound by the integrase¹⁰. The hairpin is formed by two stems separated by an unpaired region called the Unpaired Central Spacer (UCS). The apical stem is terminated by a bulge called Variable Terminal Structure (VTS), which can be of variable size and structure.

The core site of attC recombination site is composed of two integrase binding sites, named R and L boxes, which are formed by the pairing of R′ and R″, and L′ and L″ regions, respectively. The L box is 7 base pair long, located in the apical stem adjacent to the UCS. The R box is 7 base pair long, located in the stem at the base of the attC site, adjacent to the UCS.

Another important structural feature of attC sites are the Extra-Helical Bases (EHBs) located on one of the arms of the site, in the vicinity of the L″. When the site folds, these bases do not have complementary bases on the other arm, and are extruded out of the helix formed by the hairpin. While most attC sites of mobile integrons have 2 EHBs, some attC sites have 3 EHBs. One of the EHBs is located between the 4^{th} and 5^{th} base of the L″, and is usually a Guanine, even though its nature can be different. The second EHB is located closer to the apex of the attC site than the first EHB, and is usually a Thymine, even though its nature can be different. When present, the third EHB is present between the first two EHBs, and its nature can differ.

In natural attC sites, the main recombination point is located between the A and the C of the 5′-AAC-3′ triplet, located in the R′ of the bottom strand. Even though this triplet is conserved among attC sites, its sequence can be modified while keeping the high efficiency of recombination¹¹. Apart from this main recombination site in the conserved triplet of the R′ of the bottom strand, recombination can also happen in the R″ of the 5′-AAC-3′ (or derivative) of the top strand, though at lower frequency for wild-type sites ¹⁸). Also, we have described recombination in the L′ of the top strand of an attC site with the L box of an attI site in an attI×attC reaction¹². Recombination in the L″ of the bottom strand could also be possible, even though it has not been observed yet.

This disclosure provides a synthetic integron system that may be used for shuffling genes according to methods of this disclosure, for instance for the construction and optimization of metabolic pathways¹³. The invention allows for use of the integron system to rapidly create large combinatorial libraries of genetic constructs in vivo. In particular, the invention provides, among other things, compositions and methods of using the integron integrase-mediated recombination to generate in vitro or in vivo combinatorial libraries of protein-encoding genes by changing the order of gene fragments corresponding, for example, to protein domains.

This invention provides synthetic attC recombination sites. In some embodiments the synthetic attC recombination site is a DNA with the following properties:

1. It is a nucleic acid consisting of a sequence SEQ ID NO: 1 (in the 5′ to 3′ orientation) of formula

N1-N2-N3-N4-N5-N6-N7-N8-N9-N10-N11-N12-N13-N14-N15-N16-N17-N18, or a nucleic acid consisting of a sequence that is the reverse-complementary strand of a sequence (in the 5′ to 3′ orientation) of formula N1-N2-N3-N4-N5-N6-N7-N8-N9-N10-N11-N12-N13-N14-N15-N16-N17-N18, or a double stranded nucleic acid consisting of a sequence (in the 5′ to 3′ orientation) of formula N1-N2-N3-N4-N5-N6-N7-N8-N9-N10-N11-N12-N13-N14-N15-N16-N17-N18 and its complement;

2. It is a nucleic acid that can, when in single stranded form, be shifted by a purified integron integrase in an EMSA (electrophoretic mobility shift assay) with an affinity of 100 nM or higher, as described, meaning that this single-stranded DNA polynucleotide is recognized and bound by the integrase as an attC site; and

3. It is a nucleic acid which, upon integration into a vector and recombined through a suicidal conjugation assay as described in the examples, leads to recombination frequencies of 1E-6 or higher;

wherein:

N1 is 0-10 nt long;

N2 is 4 nt long and at least the last 3 nt of N2 are reverse-complementary to the first 3 nt of N17 (for example, if N2 is xAAC, then N17 has to be GTTx, x representing any nucleotide);

N3 is 5-8 nt long and it is not reverse-complementary to N16, even though upon the formation of the intramolecular imperfect hairpin, some pairings between the bases of N3 and N16 are possible;

N4 is 2-4 nt long;

N5 is 2-4 nt long;

N6 is 2-4 nt long;

N7 is from 3 nt to 30 nt long;

N8 is from 3 nt to 100 nt long;

N9 is from 3 nt to 30 nt long;

N10 is present or absent, if present then it is one of the “Extrahelical bases”;

N11 is 2-4 nt long;

N12 can be present or absent, if present then it is one of the “Extrahelical bases”;

N13 is from 2 to 4 nt long;

N14 is present or absent; if present, then it is one of the “Extrahelical bases”;

N15 is from 2 to 4 nt long;

N16 is preferentially from 5 to 8 nt long, it is not reverse-complementary to N3, even though upon the formation of the intramolecular imperfect hairpin, some pairings between the bases of N3 and N16 are possible;

N17 is 4 nt long, and at least the first 3 nt of N17 are reverse-complementary to the last 3 nt of N2 (for example, if N17 is GTTx, then N2 has to be xAAC, x representing any nucleotide); and

N18 is 0-10nt long.

In some embodiments N1 is from 0 to 10 nt long. In some embodiments N1 is reverse-complementary to N18.

In some embodiments N2 is 4 nt long. In some embodiments the last 3 nucleotides of N2 are AAC. In some embodiments at least the last 3 nt of N2 are reverse-complementary to the first 3 nt of N17 (for example, if N2 is xAAC, then N17 has to be GTTx, x representing any nucleotide).

In some embodiments N3 is from 5 to 6 nt long. In some embodiments N3 is up to 8 nt long. In some embodiments N3 it is not reverse-complementary to N16. In some embodiments, upon the formation of the intramolecular imperfect hairpin, some pairings between the bases of N3 and N16 occur.

In some embodiments N4 is 3 nt long. In some embodiments N4 is from 2 to 4 nt long. In some embodiments N4 is reverse-complementary to N15.

In some embodiments N5 is 3 nt long. In some embodiments N5 is from 2 to 4 nt long. In some embodiments N5 is reverse-complementary to N13.

In some embodiments N6 is 3 nt long. In some embodiments N6 is from 2 to 4 nt long. In some embodiments N6 is reverse-complementary to N11.

In some embodiments N7 is 3 nt long. In some embodiments N7 is up to 30 nt long. In some embodiments N7 is reverse-complementary to N9.

In some embodiments N8 is at least 3 nt long. In some embodiments N8 is up to up to 100 nt long. In some embodiments N8 is from 3 to 100 nt long.

In some embodiments N9 is at least 3 nt long. In some embodiments N9 is up to 30 nt long. In some embodiments N9 is from 3 to 30 nt long. In some embodiments N9 is reverse-complementary to N7.

In some embodiments N10 is present. In some embodiments N10 is absent. If present, N10 is one of the “Extrahelical bases”. In some embodiments N10 is thymine.

In some embodiments N11 is 3 nt long. In some embodiments N11 is from 2 to 4 nt long. In some embodiments N11 is reverse-complementary to N6.

In some embodiments N12 is present. In some embodiments N12 is absent. If present, then N12 is one of the “Extrahelical bases”. In some embodiments N12 is thymine.

In some embodiments N13 is 3 nt long. In some embodiments N13 is from 2 to 4 nt long. In some embodiments N13 is reverse-complementary to N5.

In some embodiments N14 is present. In some embodiments N14 is absent. If present, N14 is one of the “Extrahelical bases”. In some embodiments N14 is guanine.

In some embodiments N15 is 3 nt long. In some embodiments N15 is from 2 to 4 nt long. In some embodiments N15 is reverse-complementary to N4.

In some embodiments N16 is 5 nt long. In some embodiments N16 is up to 8 nt long. In some embodiments N16 is not reverse-complementary to N3. In some embodiments, upon the formation of the intramolecular imperfect hairpin, some pairings between the bases of N3 and N16 occur.

In some embodiments N17 is 4 nt long. In some embodiments the first 3 nucleotides of N17 are GTT. In some embodiments at least the first 3 nt of N17 are reverse-complementary to the last 3 nt of N2 (for example, if N17 is GTTx, then N2 has to be xAAC, x representing any nucleotide).

In some embodiments N18 can be 0-10 nt long. In some embodiments N18 is reverse-complementary to N1.

In some embodiments the synthetic attC recombination site is defined by the formula (in the 5′ to 3′ orientation) N1-N2-N3-N4-N5-N6-N7-N8-N9-N10-N11-N12-N13-N14-N15-N16-N17-N18, wherein:

N1 is 0-10 nt long and reverse-complementary to N18;

N2 is 4 nt long, its last 3 nucleotides are AAC, and at least the last 3 nt of N2 are reverse-complementary to the first 3 nt of N17 (for example, if N2 is xAAC, then N17 has to be GTTx, x representing any nucleotide);

N3 is 5-6 nt long and it is not reverse-complementary to N16, even though upon the formation of the intramolecular imperfect hairpin, some pairings between the bases of N3 and N16 are possible;

N4 is 3 nt long and it is reverse-complementary to N15;

N5 is 3 nt long and is reverse-complementary to N13;

N6 is 3 nt long and it is reverse-complementary to N11;

N7 is at least 3 nt long, and it is reverse-complementary to N9;

N8 is at least 3 nt long;

N9 is at least 3 nt long and it is reverse-complementary to N7;

N10 is present, it is one of the “Extrahelical bases,” and it is a thymine;

N11 is 3 nt long and it is reverse-complementary to N6;

N12 can be present, it is one of the “Extrahelical bases,” and it is a thymine;

N13 is 3 nt long and it is reverse-complementary to N5;

N14 is present, it is one of the “Extrahelical bases,” and it is a guanine;

N15 is 3 nt long and it is preferentially reverse-complementary to N4;

N16 is 5 nt long and it is not reverse-complementary to N3, even though upon the formation of the intramolecular imperfect hairpin, some pairings between the bases of N3 and N16 are possible;

N17 is 4 nt long, its first 3 nucleotides are GTT, and at least the first 3 nt of N17 are reverse-complementary to the last 3 nt of N2 (for example, if N17 is GTTx, then N2 has to be xAAC, x representing any nucleotide); and

N18 is 0-10 nt long and is reverse-complementary to N1.

In some embodiments the synthetic attC recombination site is defined by the formula (in the 5′ to 3′ orientation) N1-N2-N3-N4-N5-N6-N7-N8-N9-N10-N11-N12-N13-N14-N15-N16-N17-N18, wherein:

N1 can be 0-10 nt long and is preferentially reverse-complementary to N18, but does not have to be;

N2 is 4 nt long, its last 3 nucleotides are preferentially AAC, but their sequence can also be different, as we have previously shown that this conserved triplet can be modified, if corresponding changes are also made in the other recombination site participating in the recombination reaction¹¹; at least the last 3 nt of N2 are reverse-complementary to the first 3 nt of N17 (for example, if N2 is xAAC, then N17 has to be GTTx, x representing any nucleotide);

N3 is preferentially 5-6 nt long, but it can be up to 8 nt long; it is not reverse-complementary to N16, even though upon the formation of the intramolecular imperfect hairpin, some pairings between the bases of N3 and N16 are possible;

N4 is preferentially 3 nt long, but can be 2-4 nt long; it is preferentially reverse-complementary to N15;

N5 is preferentially 3 nt long, but can be 2-4 nt long; it is preferentially reverse-complementary to N13;

N6 is preferentially 3 nt long, but can be 2-4 nt long; it is preferentially reverse-complementary to N11;

N7 is at least 3 nt long, but can be up to 30 nt long; it is preferentially reverse-complementary to N9;

N8 is at least 3 nt long, but can be up to 100 nt long;

N9 is at least 3 nt long, but can be up to 30 nt long; it is preferentially reverse-complementary to N7;

N10 is preferentially present, but does not have to be; if present, then it is one of the “Extrahelical bases” and it is preferentially a thymine, even though it can be another nucleotide;

N11 is preferentially 3 nt long, but can be 2-4 nt long; it is preferentially reverse-complementary to N6;

N12 can be present or not; if present, then it is one of the “Extrahelical bases” and it is preferentially a thymine, even though it can be another nucleotide;

N13 is preferentially 3 nt long, but can be 2-4 nt long; it is preferentially reverse-complementary to N5;

N14 is preferentially present, but does not have to be; if present, then it is one of the “Extrahelical bases” and it is preferentially a guanine, even though it can be another nucleotide;

N15 is preferentially 3 nt long, but can be 2-4 nt long; it is preferentially reverse-complementary to N4;

N16 is preferentially 5 nt long, but it can be up to 8 nt long; it is not reverse-complementary to N3, even though upon the formation of the intramolecular imperfect hairpin, some pairings between the bases of N3 and N16 are possible;

N17 is 4 nt long, its first 3 nucleotides are preferentially GTT, but their sequence can also be different, as we have previously shown that this conserved triplet can be modified, if corresponding changes are also made in the other recombination site participating in the recombination reaction¹¹; at least the first 3 nt of N17 are reverse-complementary to the last 3 nt of N2 (for example, if N17 is GTTx, then N2 has to be xAAC, x representing any nucleotide); and

N18 can be 0-10 nt long and is preferentially reverse-complementary to N1, but does not have to be.

In some embodiments the synthetic attC recombination site is within a deoxyribonucleic acid (DNA) molecule, preferably within a vector or chromosome.

In some embodiments the synthetic attC recombination site comprises at least one artificial nucleotide or molecule able to mimic the properties of a nucleotide. Examples of naturally occurring nucleotides include adenosine, thymine, guanine and cytosine. Examples of artificial nucleotides include isoguanine, isocytosine, 2-amino-6-(2-thienyl) purine, pyrrole-2-carbaldehyde, 2′-deoxyinosine (hypoxanthine deoxynucleotide) derivatives. Examples of molecules that can mimic the properties of nucleotides include metal coordinated bases, such as two 2,6-bis(ethylthiomethyl)pyridine (SPy) with a silver ion or pyridine-2,6-dicarboxamide (Dipam) and a monodentate pyridine (Py) with copper ions, nitroazole analogs and hydrophobic aromatic non-hydrogen-bonding bases.

Nucleotides of the synthetic attC recombination site may take part in pairing that is not only Watson-Crick pairings such as pairings between a guanine and a cytosine or between an adenosine and a thymine, but also non-Watson-Crick pairings such as between a guanine and a thymine, between a 2-amino-8-(2-thienyl)purine and a pyridine-2-one, and others.

In some embodiments, the synthetic attC recombination site has a size of, or less than, 66, 67, 68, 69, 70, 71, 72, 72, 73, 74, 75, 80, 85, 90, 95, 100, 120 150, 200 or 300 nucleotides.

In a preferred embodiment, the synthetic attC recombination site has a sequence chosen in the group consisting of SEQ ID NO: 3 to SEQ ID NO: 16 and SEQ ID NO: 90 to SEQ ID NO: 107.

Here, the properties of synthetic attC recombination sites necessary for efficient recombination in the R′ of the bottom strand are disclosed. However, it is also possible, in certain embodiments, to modify these properties in order to shift the preferential recombination point toward the R″ of the top strand (for example, by re-localizing the extrahelical bases to the other arm of the site, meaning to the R′-L′ arm instead of the R″-L″ arm; or for example by modifying the sequence of other unpaired structures of the site, namely the Unpaired Central Spacer and the Variable Terminal

Structure). It is also possible, in certain embodiments, to these properties in order to shift the preferential recombination point toward the L′ of the top strand or L″ of the bottom strand (for example, by re-localizing the extrahelical bases from the vicinity of the L box to the vicinity of the R box; or for example by modifying the sequences of other unpaired structures of the site, namely the Unpaired Central Spacer and the Variable Terminal Structure). In this case, even though the initial properties of the polynucleotide described as N1 -N2-N3 -N4-N5-N6-N7-N8-N9-N10-N11-N12-N13-N14-N15-N16-N17-N18, would change, it would still be possible to use such sites for the applications described here, using the methods proposed here.

As a way of testing whether a DNA has the properties described as N1-N2-N3-N4-N5-N6-N7-N8-N9-N10-N11-N12-N13-N14-N15-N16-N17-N18, it is possible to predict the possible secondary structures of this polynucleotide, and verify whether they correspond to the abovementioned description. Such predictions may be performed using ViennaRNA package¹⁷, mfold package¹⁹ or other prediction software. It is important to note that even if the MFE (minimal free energy) structure does not have the abovementioned properties, it is essential, in some embodiments, to verify that at least one of the suboptimal structures (at least in the range of 10 kcal/mol above the ΔG of the MFE structure) has the abovementioned properties.

In some embodiments a synthetic attC recombination site is embedded into a genetic element of interest, such as into an open reading frame or adjacent to an open reading frame. For instance, to be used for protein domain shuffling, the synthetic attC recombination site may be embedded into an open reading frame. Exemplary non-limiting strategies for this embedding are provided herein.

Here, by integron integrase we mean any protein that can be matched with HMM (Hidden Markov Model) profiles corresponding to the presence of Tyrosine-recombinase domain and integron integrase-specific I2 domain, for example as it has been used by IntegronFinder algorithm. (Cury, J., Jové, T., Touchon, M., Néron, B. & Rocha, E. P. Identification and analysis of integrons and cassette arrays in bacterial genomes. Nucleic Acids Research 44, 4539-4550 (2016).) The HMM profiles used are the following. For Tyrosine-recombinase, Pfam Phage_integrase profile (PF00589): http://pfam.xfam.org/family/Phage_integrase. For C-terminal integron integrase domain including the I2 domain, intI_Cterm profile described in Cury et al. 2016:

http://www.ncbi.nlm.nih.gov/pmc/articles/PMC4889954/bin/supp_gkw319_nar-03309-n-2015-File011.txt. In some embodiments the IntI1 integrase of mobile integrons is used. The sequence of this IntI1 integrase is available on NCBI with the ID 6276042. However, in alternative embodiments another integron integrase, such as IntIA from Vibrio cholerae, or any other integron integrase from a mobile or chromosomal integron may be used.

### D. Recombinant Vectors, Chromosomes, and Cells

The present invention also provides recombinant vectors and recombinant chromosomes comprising at least one protein coding sequence of a multidomain protein, wherein the protein coding sequence comprises at least one synthetic attC recombination site. In some embodiments the recombinant vector or recombinant chromosome comprises a plurality of synthetic attC recombination sites. In some embodiments at least one synthetic attC recombination site has a size of 73 nucleotides or less. In some embodiments the multi-domain protein is a modular type I polyketide synthase (PKS). In some embodiments the multi-domain protein is a non-ribosomal polypeptide synthase (NRPS).

The present invention also provides recombinant cells comprising at least one nucleic acid comprising a protein coding sequence of a multidomain protein, wherein the protein coding sequence comprises at least one synthetic attC recombination site. In some embodiments the recombinant nucleic acid is a DNA. In some embodiments the recombinant nucleic acid is a vector. In some embodiments the recombinant nucleic acid is a chromosome. In some embodiments the recombinant cell comprises a plurality of nucleic acids comprising a protein coding sequence of a multidomain protein, wherein the protein coding sequence comprises at least one synthetic attC recombination site. In some embodiments the recombinant cell comprises a plurality of vectors comprising a protein coding sequence of a multidomain protein, wherein the protein coding sequence comprises at least one synthetic attC recombination site. In some embodiments the recombinant cell comprises a plurality of chromosomes comprising a protein coding sequence of a multidomain protein, wherein the protein coding sequence comprises at least one synthetic attC recombination site. In some embodiments the recombinant cell comprises a plurality of vectors and a plurality of chromoromses, each comprising a protein coding sequence of a multidomain protein, wherein the protein coding sequence comprises at least one synthetic attC recombination site. In some embodiments each of the vector(s) and/or chromosome(s) present in the cell comprises a plurality of protein coding sequences of a multidomain protein, wherein each protein coding sequence comprises at least one synthetic attC recombination site. In some embodiments the at least one synthetic attC recombination site has a size of 73 nucleotides or less. In some embodiments the multi-domain protein is a modular type I polyketide synthase (PKS). In some embodiments the multi-domain protein is a non-ribosomal polypeptide synthase (NRPS). In some embodiments the recombinant nucleic acid comprises at least one recombined synthetic attC recombination site.

### E. Embedding Synthetic attC Recombination Sites Into Genetic Elements of Interest

An exemplary strategy for embedding synthetic attC recombination sites into genetic elements of interest is the following.

1. A region in the DNA sequence of the element of interest is chosen for embedding of a synthetic attC recombination site. The localization of the recombination point is also chosen.

2. Various synthetic attC recombination sites (number=N) are then generated, each having the sequence N1-N2-N3-N4-N5-N6-N7-N8-N9-N10-N11-N12-N13-N14-N15-N16-N17-N18. In some embodiments, at this step each synthetic attC recombination site is not a single sequence, but an ensemble of two sequences, N1-N2-N3-N4-N5-N6-N7 and N9-N10-N11-N12-N13-N14-N15-N16-N17-N18, separated by a region of undefined length N8. In some embodiments additional constraints are introduced at this step, such as those described below, or identified with the approaches described below.

3. For each of the N synthetic attC recombination sites, all or a subset of possible positions where this site could be embedded are considered, and a score of such embedding is calculated. For example, this score can be calculated by comparing the region described in [00157] and the sequence N1-N2-N3-N4-N5-N6-N7-N8-N9-N10-N11-N12-N13-N14-N15-N16-N17-N18, and attributing a positive value to each property or element that is deemed important for the functionality of region described in [00157] and that is present in the sequence N1-N2-N3-N4-N5-N6-N7-N8-N9-N10-N11-N12-N13-N14-N15-N16-N17-N18. Such property or element can for example be: the GC content of the region; a particular sequence, such as a GATC methylation site; a particular ΔG value of the folded sequence as predicted by a nucleic acid structure prediction software; a particular amino-acid sequence upon translation; etc.

4. A set of sites with the best scores are selected, such as for example the 0.5N top sites.

5. Each of the selected sites is mutated, and compensatory mutations are introduced if needed, to maintain the properties of the sequence N1-N2-N3-N4-N5-N6-N7-N8-N9-N10-N11-N12-N13-N14-N15-N16-N17-N18.

6. The library comprising the initial and the mutated set of sequences is re-submitted for consecutive steps 3, 4 and 5, for a plurality of cycles.

7. The top-scoring site(s) from the final library are retained as the result. In some embodiments a different scoring system is then implemented at this point. In some embodiments additional constraints are implemented at this point, or identified with the approaches described below. At this step the selected site(s) may be used directly, or may be subjected to functional testing before moving forward.

### F. Embedding Synthetic attC Recombination Sites Into Protein-Coding Regions

The invention encompasses the following strategy for embedding synthetic attC recombination sites into protein-coding regions.

1. A region in the DNA sequence of the element of interest is chosen, where the synthetic attC recombination site is to be embedded. Also, the localization of the recombination point is chosen. When using these synthetic attC recombination sites for protein domain shuffling, the location of the recombination points is chosen in a way that upon embedding of a plurality of synthetic attC recombination sites and subsequent recombination, the open reading frame is preserved.

2. Various synthetic attC recombination sites (number=N) are generated, each one of them having the sequence N1-N2-N3-N4-N5-N6-N7-N8-N9-N10-N11-N12-N13-N14-N15-N16-N17-N18. In some embodiments, at this step each synthetic attC recombination site is considered not as a single sequence, but as an ensemble of two sequences N1-N2-N3-N4-N5-N6-N7 and N9-N10-N11-N12-N13-N14-N15-N16-N17-N18, separated by a region of undefined length N8. In some embodiments additional constraints are introduced at this step, such as those described below, or identified with the approaches described below.

3. For each of the N synthetic attC recombination sites, all or a subset of possible positions where this site could be embedded are considered, and a score of such embedding is calculated. When using these synthetic attC recombination sites for protein domain shuffling, in some embodiments the position of N9-N10-N11-N12-N13-N14-N15-N16-N17-N18 is held constant, and the position of the N1-N2-N3-N4-N5-N6-N7 moiety is changed. Then, a score of such embedding is calculated. For example, this score can be calculated using a substitution matrix such as BLOSUM (for example, BLOSUM62) or PAM (for example, PAM30 or PAM70). This score can be obtained by comparing the translation of the region described in

in the frame corresponding to its open reading frame of the element of interest, and the translation of the sequence N1-N2-N3-N4-N5-N6-N7-N8-N9-N10-N11-N12-N13-N14-N15-N16-N17-N18 in all possible frames in the same sense as to its open reading frame of the element of interest, where N8 is either defined at step 2, or corresponds to a region of the sequence described in [00165]. The total score is compared by adding the values given by the substitution matrix for each amino acid of the region described in [00165], with its counterpart in the sequence N1-N2-N3-N4-N5-N6-N7-N8-N9-N10-N11-N12-N13-N14-N15-N16-N17-N18.

4. A set of sites with the best scores are selected, such as for example the 0.5N top sites.

5. Each of the selected sites is mutated, and compensatory mutations are introduced if needed, to maintain the properties of the sequence N1-N2-N3-N4-N5-N6-N7-N8-N9-N10-N11-N12-N13-N14-N15-N16-N17-N18.

6. The library comprising the initial and the mutated set of sequences is re-submitted for consecutive steps 3, 4 and 5, for a plurality of cycles.

7. The top-scoring site(s) from the final library are retained as the result. In some embodiments a different scoring system is then implemented at this point. In some embodiments additional constraints are implemented at this point, or identified with the approaches described below. At this step the selected site(s) may be used directly, or may be subjected to functional testing before moving forward.

The invention further encompasses a method comprising selecting a protein coding sequence and inserting at least one synthetic attC recombination site into the protein coding sequence to generate a protein coding sequence comprising a synthetic attC recombination site. Within the scope of this invention, a protein coding sequence comprising a synthetic attC recombination site is not found in nature.

Preferably the protein coding sequence comprising at least one synthetic attC recombination site is in a vector, most preferably, a plasmid vector.

In a preferred embodiment, the invention encompasses a method comprising selecting a protein coding sequence in a plasmid and inserting a DNA comprising at least one synthetic attC recombination site into the protein coding sequence of the plasmid to generate plasmid comprising a protein coding sequence comprising a synthetic attC recombination site.

In some embodiments, the protein coding sequence comprises at least two, three, four, five, six, seven, eight, nine, ten, fifteen, twenty, twenty-five, or thirty synthetic attC recombination sites.

Preferably, the protein coding sequence is of a multidomain protein. Most preferably, the protein is a polyketide synthase or a non-ribosomal polypeptide synthetase.

The invention further encompasses the nucleic acids produced by the method and the proteins encoded by the nucleic acids.

### G. Optional Constraints for Synthetic attC Recombination Sites

In some embodiments additional constraints or scoring systems for synthetic attC recombination sites are incorporated. For example, additional constraints or scoring systems may be incorporated at the step of generating synthetic attC recombination sites, or at the step of selection of best candidates after embedding them into genetic elements of interest. Non-limiting examples of the constraints and scorings, and of approaches aimed at identifying such constraints and scoring principles, include following:

1. Imposing a non-arbitrary composition in nucleotides of the unpaired regions, namely in the Unpaired Central Spacer, and the Variable Terminal Structure: for example, imposing a higher content in purines in the bottom strand of the attC site¹⁸.

2. Imposing a non-arbitrary composition in nucleotides of the paired regions, namely in apical and the basal stems: for example, imposing a lower GC content in the apical stem of the attC site (Grieb M. S., Nivina A., Cheeseman B. L., Hartmann A., Mazel D., Schlierf M. Nucleic Acids Research, 2017 (in print)).

3. Imposing a non-symmetric Unpaired Central Spacer, for example when N3 is 6 nt long and N16 is 5 nt long.

4. Imposing low positional entropies (<1 or <1.5) for the Extra-Helical

Bases. Positional entropies for the extra-helical bases can be calculated using one of the secondary structure prediction softwares, such as ViennaRNA package¹⁷.

5. Imposing that N5 and N6 together are 6nt long, and that N11 and N14 together are 6 nt long.

6. Imposing that N4 and N15 are 4 nt long.

3. Identifying additional constraints and/or scoring principles by performing high-throughput testing of libraries of synthetic attC recombination sites as described below, and using the resulting data either to deduce the constraints that beneficial for high performance of the attC sites in recombination; such as to train a Machine Learning algorithm and use it to predict the recombination frequency of synthetic attC recombination sites and to deduce the important features, as described below.

### H. Using Synthetic attC Recombination Sites for Protein Domain Shuffling in Polyketide Synthases, Non-Ribosomal Polypeptide Synthetases, or Other Multidomain Proteins

Modular type I polyketide synthases (PKS) are large multi-enzyme systems of multi-domain proteins, involved in the synthesis of secondary metabolites. Some of these metabolites or their derivatives are used in medicine as antibiotics, immunosuppressants, chemotherapies and other therapeutic classes¹⁴. These systems assemble carbon chain backbones of polyketides from two-, three-, and four-carbon building blocks such as acetyl-CoA, propionyl-CoA, and butyryl-CoA and their activated derivatives in a series of elongation steps. Each of these chain elongation steps is performed by a different module of the PKS, the resulting molecule being transferred from one module to the next one, until it is (usually) circularized and released. After each of the elongation steps, one or several functional group modifications (such as ketoreduction, dehydration, enoyl reduction, and others) can be performed either by the module itself or by an enzyme expressed in trans.

Non-ribosomal polypeptide synthases (NRPS) are another class of modular secondary metabolite synthesis proteins. Also organized in modules, they polymerase amino acids and generate a large number of secondary metabolites, some of them being used as therapeutics, such as antibiotics. Different NRPS enzymes are capable of using both the 21 proteogenic amino acids, as well as a number of non-standard amino acids. Within each module, these precursors are activated by the adenylation domain, loaded onto the Peptidyl Carrier Protein domain and condensed with the nascent molecule carried by the previous module. As for PKS, additional modifications can occur either by domains located within the module, or by enzymes expressed in trans. Also, hybrid PKS/NRPS secondary metabolite biosynthesis systems are possible.

This invention provides methods of shuffling modules of these proteins to produce novel PKSs and/or NRPSs that synthesize novel molecules. Embedding synthetic attC sites into PKS and/or NRPS genes and shuffling the modules allows production of combinatorial libraries of novel proteins in vivo, which may then be screened for the production of molecules of therapeutic interest. The feasibility of the assembly of novel PKSs by re-arranging the existing modules has been previously shown^{15,16}. However, the compositions and methods of this invention rely on the embedding of recombination sites into protein-coding DNA sequence of PKS and/or NRPS and performing high-throughput combinatorial shuffling in vivo, via integron integrase-mediated DNA recombination. This approach is particularly useful because it allows for the creation and screening of PKS and/or NRPS variants on a large scale.

In order to perform protein domain shuffling in polyketide synthases, non-ribosomal synthetases or other multidomain proteins, we propose the following approach may be used. For illustrative purposes only, this example is described in reference to PKS.

1. In a first module of a PKS, select a region that is deemed to be relatively less important for the functionality of the PKS. For example, the linker regions between the adjacent domains within the modules (for example, the region between the ketosynthase domain and the acyltransferase domain; the region between the acyltransferase domain and the ketoreductase or dehydratase domain; the region between the ketoreductase domain and the acyl carrier protein domain; etc.), or the linker region connecting the two adjacent modules can be chosen.

2. In one or several other PKS modules, select similar region(s). In these regions, select a recombination point in a way that upon recombination and reshuffling of modules, the open reading frame(s) would be reconstituted. Preferentially, these recombination points are located in the patches of relative homology between the selected regions, so that upon recombination open reading frames are reconstituted and there is a reasonable likelihood of reconstituting functional modules.

3. Embed synthetic attC recombination sites into each of the selected regions using the approach described above. Several candidate synthetic attC recombination sites can be retained for each of the modules and optionally may be tested in parallel.

4. It is possible to evaluate the performance of candidate synthetic attC recombination sites to select those that have higher recombination frequencies and/or those that are least disruptive of the activity of the PKS, using for example the approaches described below.

5. Construct the platform for shuffling modules, by (1) replacing the initial regions by the sequences of the selected synthetic attC recombination sites, either on a chromosome or on another vector; and (2) inducing the shuffling of modules by expressing the integron integrase. An exemplary method of constructing such a shuffling platform is described below.

### I. Constructing a Shuffling Platform for Generating Combinatorial Libraries of Polyketide Synthases, Non-Ribosomal Polypeptide Synthetases, or Other Multidomain Proteins

An exemplary preferred embodiment of the platform used for generating combinatorial libraries of polyketide synthases, non-ribosomal polypeptide synthetases, or other multidomain proteins, is the following.

A vector, or a chromosome, containing the DNA fragment corresponding to the “starting platform” is provided. In case of combinatorial assembly of novel PKSs, the system may contain the “starting platform” containing at least the following: the loading domain and the beginning of the first module, at least one synthetic attC recombination site, as well as the end of the last module, and optionally the thioesterase domain. This “starting platform” may optionally also include a promoter and a ribosome binding site upstream of the elements described above, so as to allow the expression of the resulting PKS. This expression could be either constitutive or inducible, depending on the promoter chosen.

Either the same vector or chromosome, or at least one additional vector and/or chromosome, may also be provided containing “units” of the combinatorial assembly, each flanked by synthetic attC recombination sites embedded into its original sequence. In case of combinatorial assembly of novel PKSs, each of these units may correspond to a fragment of a PKS gene roughly of the size of a module or a domain, and flanked by synthetic attC recombination sites, in such a way that upon the insertion of such cassettes into the “starting platform”, this would reconstitute whole PKS modules.

The vectors and/or chromosomes are combined in vitro or in vivo with an integron integrase to catalyze recombination. When the vectors and/or chromosomes are combined in a cell an integron integrase gene is also introduced into the cell and expressed so as to induce recombination. In some embodiments an integron integrase open reading frame is operatively linked to an inducible promoter to allow control of expression of the integrin integrase and thus control of recombination in the system.

### J. Performing Screening of the Resulting Combinatorial Libraries of Novel PKS, NRPS or Other Multimodular Enzymes

In order to be able to select the shuffled PKS, NRPS or other multidomain proteins that have the desired properties, the following exemplary approach may be used.

The aforementioned shuffling platform is constructed in the host organism, which is either a bacterium (such as Escherichia coli), a fungus (such as a yeast Saccharomyces cerevisiae), another microorganism, or even a synthetic cell, micelle or droplet capable of transcribing and/or translating information encoded by nucleic acid.

First, a shuffling step is performed and a library of host organisms is obtained, with theoretically each cell having on average about one different PKS generated in the platform, and thus potentially capable of synthesizing a different secondary metabolite. This population of host organisms is then encapsulated in micelles, droplets, capsules or other particles capable of containing cells and being handled in a microfluidic device. This encapsulation may proceed in such a way that only one host organism is encapsulated per particle, on average, so that during the screening process each particle corresponds to one genotype, and consequently one phenotype. However, in some embodiments to incorporate the cells into particles first, and perform the shuffling step once the cells are encapsulated.

Also, one is to incorporate a reporter system into the same particle, depending on the screening that one wishes to perform. Examples of reporter systems are the following: another bacterial cell (for screening for antibiotics and antibacterials); a fungal cell (for screening for antifungal molecules); a eukaryotic cell (for screening for molecules having effect on human or animal diseases); a cancer cell (for screening for cancer chemotherapies); a synthetic cell designed to function as a reporter of a condition. Such reporter systems should be labeled in such a way that a detection system (such as a microscope) would be able to distinguish between the reporter cells presenting the sought effect (such as killing of the reporter cell, inhibiting its growth, activation of a certain pathway in a reporter cell, etc.) and the other reporter cells. It is also conceivable to perform these tests without using reporter cells, and using the host cells themselves as reporters, for example for screening for molecules active against the host organism itself

These particles are later screened for a desired phenotype of the reporter cells, and separated depending on this phenotype. The selected particles are then analyzed to identify the sequence of the PKS, NRPS or other multidomain protein that resulted in the sought phenotype. These novel genes and the resulting proteins are then considered as candidates for the production of novel therapeutic molecules.

### EXAMPLES

### Example 1: Materials and Methods

### A. Suicidal Conjugation Assay

The suicidal conjugation assay for measuring the recombination frequencies of attC sites is based on the previously described⁸ protocol. It consists of delivering one strand of a plasmid, carrying either the top or the bottom strand of an attC site, into a recipient strain capable of performing an attI1×attC reaction. The donor strain β2163 requires DAP (diaminopimalic acid) to grow in rich medium, and contains the RP4 conjugative transfer system and the pir gene on its chromosome. The latter allows the replication of a pSW23T plasmid with a pir-dependent origin of replication oriVR6Kγ19, which carries an RP4 transfer origin, an attC site and a chloramphenicol resistance marker. Depending on the pSW23T-based vector used for cloning, the strand transferred by β2163 during conjugation carries either a bottom strand of the attC site (p4116 as vector) or a top strand (p4117 as vector). The recipient DH5α strain expresses the IntI1 integrase from a pBAD plasmid (p3938) and carries a pSU38Δ plasmid containing an attI1 site (p929), but is unable to maintain the replication of the pir-dependent pSW23T plasmid that is transferred into the recipient by conjugation. The only way to maintain its replication and express the encoded chloramphenicol resistance marker is to integrate pSW23T as a cassette into the pSU38Δ plasmid through attI1×attC recombination.

For this assay, the donor and the recipient strains are grown overnight with the corresponding antibiotics and other chemicals (Km, Cm and DAP for the donor; Km, Carb and Glc for the recipient), then diluted 1:100 with the corresponding antibiotics and other chemicals (Km and DAP for the donor; Km and Ara for the recipient) and grown until the OD600=0.4 to 0.5. Then, 1 ml of donor and 1 ml of recipient cultures are mixed, centrifuged, and the pellet is spread on a 0.45 μm filter placed on a LB-agarose media supplemented with DAP and Ara, for an overnight conjugation. The filter is then resuspended in 5 ml LB media, after which serial 1:10 dilutions are made, and 100 μl are plated on LB-agarose media supplemented with Cm and on LB-agarose media supplemented with Km. The recombination frequency is calculated as the ratio of recombinant CFUs [CmR] to the total number of recipient CFUs [KmR].

### B. Electrophoretic Mobility Shift Assay (EMSA)

This electrophoretic mobility shift assay (EMSA) allows the determination whether the single-stranded DNA molecule corresponding to the tested sequence is recognized and bound by the integron integrase, as an attC site.

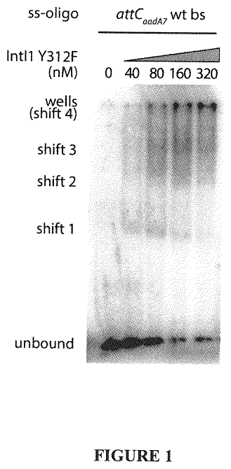

Each reaction contains 500 ng Poly[d(I-C)], 12 mM Hepes-NaOH pH 7.7, 12% glycerol, 4 mM Tris-HCl pH 8.0, 60 mM KCl, mM EDTA, 0.06 mg/ml BSA, 1 mM DTT, 10% Tween 20, 0.5 pmol of the corresponding ³²P-labeled DNA oligonucleotide and approximately 25 ng (1 pmol) of purified integron integrase (either active integrase, or inactive mutant with its catalytic Tyrosine mutated for example to Phenylalanine; also, possible to use equivalent quantities of integrase expressed with a tag, such as His-tag or MBP-tag) in a final volume of 20 μl. The samples are incubated at 30° C. for 10 min without the probe followed by 20 min with the probe, then loaded to a 5% native polyacrylamide gel (Acrylamide/Bisacrylamide 37.5:1), with 0.5× TBE as buffer. The gel has to be run for 2 h at 40 mA with 0.5× TBE as running buffer and visualized using chemoluminescence film (Amersham Hyperfilm™). A shift resembling that on FIG. 1 testifies that the integron integrase binds the tested oligonucleotide as it does an attC site.

### C. Library Construction and Testing

A method of constructing and testing libraries of synthetic attC recombination sites in order to infer supplementary information relative to the important features of attC sites for incorporation at the step of generation of synthetic attC recombination sites with arbitrary sequences, or for use for scoring in the process of embedding of synthetic attC recombination site into a genetic region of interest.

First, a library exploring a certain mutational space of an attC site is constructed. For this purpose, different approaches are possible: creating a library of n possible mutations (for example, for n=2, a library of all possible double mutations) in a region of the attC site by performing mutagenic PCR reactions; creating a library of n possible mutations (for example, for n=2, a library of all possible double mutations) in a region of the attC site by using degenerate primers which have a non-nil percentage of incorporation of nucleotides other than the initial sequence throughout the selected region; creating a library of all possible nucleotide combinations in particular positions by using degenerate primers which have a non-nil percentage of incorporation of nucleotides other than the initial sequence in the selected positions (for example, a 25% incorporation rate for each of the four natural nucleotides); or creating other types of libraries by performing a combination of these techniques.

These libraries are constructed by cloning the library of DNA fragments thus diversified into a vector or a chromosome. The resulting colonies are collected, mixed, and kept as the actual library.

This library then is recombined, for example through a method similar to the one described as the suicidal conjugation assay, with the ensemble of bacteria containing the library used as donor strain. After one or several rounds of such recombination experiments, the library will be enriched in clones corresponding to highly recombinogenic mutants of the attC site. This enriched library can then be sequenced, for example by amplifying the region of the vector or chromosome containing the attC site, and performing deep sequencing, such as Ion Proton,

Illumina or other techniques. In order to normalize the data, same kind of deep sequencing can be performed on the initial library (before recombination). The information about the enrichment of each individual mutant can thus be obtained and kept for further analysis.

One possibility is to identify the features of attC sites that are enriched in the libraries throughout the enrichment cycles. These features can then either be added as constraints used at the moment of generation of synthetic attC recombination sites with arbitrary sequences; or be used as an additional factor for scoring the library of synthetic attC recombination sites while embedding them into genetic regions of interest.

Another possibility is to use the resulting data to train a Machine Learning algorithm which could be used to predict the recombination frequency of synthetic attC recombination sites, as described below

### D. Use of Machine Learning Algorithm for the Prediction of Recombination Frequencies

The results of high-throughput testing approaches described above may be analyzed using Machine Learning algorithms.

First, the data used is the enrichment value, which is calculated the following way for each of the mutated sites: (Number of reads for this sequence after cycle 1/Total number of reads after cycle 1)/(Number of reads for this sequence in the initial library/Total number of reads in the initial library).

If for a certain mutant, no reads were detected, the number of reads for this mutant is set equal to 1.

Second, a set of features describing the sequence and the secondary structure of each recombination site is used. The values of these features for example can all be attributed to a value between 0 and 1. These features can include, but are not limited to: the nucleotide located in each of the mutated positions; the ΔG of the structure; the positional entropy of each of the position of the site; the probability of being paired for each of the position of the site; the probability to fold into a recombinogenic structure that has correctly paired R and L boxes; etc. For each site, the values of these features can be obtained via a folding prediction software, such as ViennaRNA (Lorenz, R. et al. ViennaRNA Package 2.0. Algorithms Mol Biol 6, 26 (2011)), and then normalized to fit into the range between 0 and 1, in order to be used by a Machine Learning algorithm.

This data may then be used to perform supervised learning, with either a classification, or a regression algorithm. Such algorithms include, but are not limited to such approaches as Linear Regression, Ridge Regression, Decision Tree regression, Random Forest, Random Forest Regression, Support Vector Machines, Support Vector Regression, Neural Networks, etc.

The performance of these algorithms can then by analysed in terms of feature importance among the features used by these algorithms. The biological interpretation of the important features can be used to deduce optional constraints for synthetic attC recombination sites, such as those mentioned in section G.

One such exemplary algorithm has been trained on a set of data obtained through a high-throughput experiment as the one described before and involving the construction and testing of libraries of mutated attC sites, this algorithm can be used in order to predict the recombination of attC sites that have not been part of the training data, and deduce optional constraints for synthetic attC recombination sites mentioned in section G.

### E. Evaluating the Performance of Selected Synthetic attC Recombination Sites in Recombination

A test for evaluating the performance of selected synthetic attC recombination sites in recombination is then applied. The purpose of this test is to test the recombination of a synthetic attC recombination site in conditions similar to those of the fully constructed system.

A system is used where the synthetic attC recombination site is followed by a cassette of a certain length, and another attC site with a known recombination frequency. This system is designed in such a way that upon recombination of the two sites, a selection marker is reconstituted, such as an antibiotic resistance gene, or an essential gene such as dapA (in the absence of which cells can grow only if supplemented with diaminopimelic acid). This system allows to determine the frequency of cassette excision events by dividing the number of colonies resistant to the marker (or not requiring supplementation with diaminopimelic acid) by the number of total colonies (or those growing with diaminopimelic acid).

Another system may also be used, where the synthetic attC recombination site is located on the vector or chromosome, and the cassette is provided on another vector, typically a plasmid. This system is designed in such a way that upon recombination of there two sites, a selection marker is reconstituted, such as an antibiotic resistance gene, or an essential gene such as dapA (in the absence of which cells can grow only if supplemented with diaminopimelic acid). This system allows to determine the frequency of cassette integration events by dividing the number of colonies resistant to the marker (or not requiring supplementation with diaminopimelic acid) by the number of total colonies (or those growing with diaminopimelic acid).

### F. Evaluating the Performance of the Genetic Element into which Synthetic attC Recombination Sites were Embedded

The performance of the genetic element into which synthetic attC recombination sites were embedded is evaluated. The principle of this assay is based on the evaluation and quantification of the activity of the protein into which the synthetic attC cites have been embedded. As the functions performed by the different proteins are different, the assays are different, too.

However, it is possible to perform general assessment assays that can inform one whether the embedding of a synthetic attC sites is detrimental for the protein. One of such assays is a Western blot, performed with an antibody directed against the protein in questions, if such is available. This assay will allow to determine whether the size and at least some of the epitopes are preserved in the modified protein. Other, more specific assays, are developed on a case-by-case basis.

The performance of PKS, NRPS or other proteins involved in the synthesis of secondary metabolites is also evaluated. An assay for such proteins could rely on the expression of these proteins together with the genes necessary for the production of necessary precursors, and the quantification of the produced secondary metabolite. If the embedded synthetic attC recombination site does not prohibit the correct functionality of the protein, the amount of secondary metabolite produced by such a system should be comparable to that produced by a wild-type gene. A general approach would be to express the genes at 22° C. for ˜3 days, and perform a purification step by an HPLC (high-throughput liquid chromatography) by eluting the compounds by a gradient of acetonitrile in water, from a hydrophobic column. However, the exact procedure of quantification might have to be adapted to each particular metabolite.

### G. Strains and Plasmids

The bacterial strains and plasmids used in these examples are listed in Table 1.

TABLE 1
Bacterial strains and plasmids.

| Strain/ | | | |
|---|---|---|---|
| plasmid | Name | Genotype or description | Source or reference |
| E. coli | MG1655 | MG1655 | Laboratory collection |
| strains | β2163 | (F2) RP4-2-Tc::Mu | Demarre, G. et al. A |
| | | dapA::(erm, pir); [Km^{R}] | new family of |
| | | | mobilizable suicide |
| | | | plasmids based on |
| | | | broad host range |
| | | | R388 plasmid (IncW) |
| | | | and RP4 plasmid |
| | | | (IncPalpha) conjugative |
| | | | machineries and their |
| | | | cognate Escherichia |
| | | | coli host strains. |
| | | | Research in |
| | | | Microbiology 156, |
| | | | 245-255 (2005). |
| | DH5α | DH5α | Laboratory collection |
| Plasmids | p4116 | pSW23T::attC_{aadA7}T23inv | Bouvier, M., Ducos- |
| | | (BOT); oriV_{R6Kγ}, oriT_{RP4}; | Galand, M., Loot, C., |
| | | [Cm^{R}] | Bikard, D. & Mazel, D. |
| | | | Structural features of |
| | | | single-stranded integron |
| | | | cassette attC sites and |
| | | | their role in strand |
| | | | selection. PLoS Genet |
| | | | 5, e1000632 (2009). |
| | p4117 | pSW23T::attC_{aad47}T23inv | Bouvier, M., Ducos- |
| | | (TOP); oriV_{R6Kγ}, oriT_{RP4}; | Galand, M., Loot, C., |
| | | [Cm^{R}] | Bikard, D. & Mazel, D. |
| | | | Structural features of |
| | | | single-stranded integron |
| | | | cassette attC sites and |
| | | | their role in strand |
| | | | selection. PLoS Genet |
| | | | 5, e1000632 (2009). |
| | p3938 | pBAD::intI1; oriColE1; | Demarre, G., Frumerie, |
| | | [Ap^{R}] | C., Gopaul, D. N. & |
| | | | Mazel, D. Identification |
| | | | of key structural |
| | | | determinants of the |
| | | | IntI1 integron integrase |
| | | | that influence attC x |
| | | | attI1 recombination |
| | | | efficiency. Nucleic Acids |
| | | | Research 35, 6475-6489 |
| | | | (2007). |
| | p929 | pSU38Δ::attI1; orip15A; | Biskri, L., Bouvier, M., |
| | | [Km^{R}] | Guerout, A.-M., |
| | | | Boisnard, S. & Mazel, |
| | | | D. Comparative study |
| | | | of class 1 integron and |
| | | | Vibrio cholerae |
| | | | superintegron integrase |
| | | | activities. Journal of |
| | | | Bacteriology 187, 1740- |
| | | | 1750 (2005). |

### Example 2: Synthetic attC Recombination Sites with Arbitrary Sequences

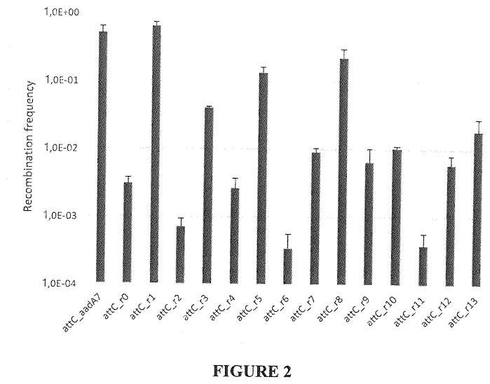

Fourteen synthetic attC recombination sites with arbitrary sequences have been constructed (Table 2). The synthetic attC recombination sites have been evaluated using the suicidal conjugation assay. The results obtained are presented in FIG. 2, and show that all these sites recombine (FIG. 2).

TABLE 2
Sequences of the naturally
occurring attC_{aadA7} site, and of
synthetic attC sites (top strands) tested.

| attC | | |
|---|---|---|
| site | SEQ ID | Sequence |
| attC_{aadA7} | SEQ ID | CGGTTATAACAATTCATTCAAGCCGACGCCGCT |
| | NO: 2 | TCGCGGCGCGGCTTAATTCAAGCGTTATAACCG |
| attCr0 | SEQ ID | GAATTCATTATAACGGAGGTTACCCATGGATTC |
| | NO: 3 | GAGTTCCTCGAACCATGGTAAAGAGTGTTATAA |
| | | TGGATCC |
| attCr1 | SEQ ID | GAATTCCGTCTAACTCATCGCGCGTGAATAAAC |
| | NO: 4 | CTCTTGGAGGTTATTCACCGCAAAATGTTAGAC |
| | | GGGATCC |
| attCr2 | SEQ ID | GAATTCAGGGTAACGCTACGCGCAGTGCCAAGC |
| | NO: 5 | ATCTATGATGCTGGCACTCGCCTCTTGTTACCC |
| | | TGGATCC |
| attCr3 | SEQ ID | GAATTCTCTCTAACCTGCTACTCTATAGTACAG |
| | NO: 6 | TAAGGTTTACTGACTATAAGTCGGGTGTTAGAG |
| | | AGGATCC |
| attCr4 | SEQ ID | GAATTCTTTCTAACTCCGCCAACCCGGAGAAGC |
| | NO: 7 | GTGGCCCACGCTCTCCGGTTGATATTGTTAGAA |
| | | AGGATCC |
| attCr5 | SEQ ID | GAATTCGCCTTAACAATACAGGCTATGTTATTT |
| | NO: 8 | GGTCGGACCAAAAACATACCTAGTAGGTTAAGG |
| | | CGGATCC |
| attCr6 | SEQ ID | GAATTCATCTTAACTGCTTACACCCGGGCAACC |
| | NO: 9 | TTTCCTAAAGGTGCCCGGTGTGGCTTGTTAAGA |
| | | TGGATCC |
| attCr7 | SEQ ID | GAATTCTCGGCTAACCGCTCAGACTATCGCACT |
| | NO: 10 | ACCTGGTTTCTAGGCGATATCTTCGGAGTTAGC |
| | | CGAGGATCC |
| attCr8 | SEQ ID | GAATTCTAGGCTAACAGAACGGTCAATATGAGG |
| | NO: 11 | GAGGCTGGATCCCCATATTACCTCGGTGTTAGC |
| | | CTAGGATCC |
| attCr9 | SEQ ID | GAATTCACGACGAACTCAGACGACAGATATAAC |
| | NO: 12 | CTAAAAGTTCGGTATATCTTCGGGCCGTGTTCG |
| | | TCGTGGATCC |
| attCr10 | SEQ ID | GAATTCCTGCCTAACGTCGTCTGCAGCGTCACA |
| | NO: 13 | CTGTACGCATGTGGACGCTCAGTCAAATGTTAG |
| | | GCAGGGATCC |
| attCr11 | SEQ ID | GAATTCTTACACAACGGCCCATACTGAATCAGA |
| | NO: 14 | AATCCAAACATTCGATTCATATTCGACGTTGTG |
| | | TAAGGATCC |
| attCr12 | SEQ ID | GAATTCATGGCTAACTAGTAATACTCAGGGAAT |
| | NO: 15 | CGATCACGGTGATCCCTGATATGCTCTGTTAGC |
| | | CATGGATCC |
| attCr13 | SEQ ID | GAATTCGCCACTAACAGTTTCTACGATTTGAAT |
| | NO: 16 | GTCGATATCGCATCAAATCTAGCGGCTCGTTAG |
| | | TGGCGGATCC |

### Example 3: Enrichment of High-Throughput Libraries of Mutated attC Sites with Arbitrary Sequences, and the Use of Machine Learning to Predict Their Recombination Frequency

The following three libraries of the attCr0 synthetic recombination site were constructed:

Library 1 represents the ensemble of all single and double mutants of attC0 throughout all the site up to the recombination point;

Library 2 represents the ensemble of all possible mutations in 8 positions located in the UCS and the R box on both arms of the site; and

Library 3 represents the ensemble of all possible mutations in 7 positions located in the VTS.

Two enrichment cycles for each of the libraries were performed. The region containing the attC site mutant was amplified before enrichment and after each of the cycles. The resulting PCR products were then sequenced by Ion Proton deep sequencing.

A Machine Learning Algorithm was applied on the sample of enrichment data for Library 1, and its performance on the rest of the data was evaluated. The classification algorithm using Random Forest was able to predict whether a particular recombination site was enriched or lost during the enrichment cycles with 84% accuracy. As the enrichment value is highly correlated to the actual recombination frequency of mutants in the library, this performance of the algorithm corresponds to its capacity to predict whether a recombination site from the library recombines well or not, based on its sequence.

A similar Machine Learning Algorithm is trained to perform regression on the same set of training and testing data.

This algorithm, or a similar one, is used in for embedding synthetic attC recombination sites into genetic regions of interest. In particular, the algorithm is used to score the candidate embedded attC sites in order to select those that have a higher chance of being highly recombinogenic, and/or selecting synthetic attC sites that recombine within a desired range.

### Example 4: Synthetic attC Recombination Sites Embedded into Protein-Coding Region (lacZ)

The approach for embedding synthetic attC recombination sites into protein-coding regions described above was used to generate four synthetic attC recombination sites embedded into the Escherichia coli lacZ gene. These experiments serve as a general proof of concept that the embedding of synthetic attC recombination sites into protein-coding regions is possible without interfering with the protein's function.

The performance of these synthetic attC recombination sites in recombination, as described above, is tested in excision and reintegration experiments.

The ability of these synthetic attC recombination sites embedded into the lacZ gene to not interfere with the functionality of the resulting β-galactosidase protein is tested by performing colorimetric assays. These assays are based on the principle of beta-galactosidase assay, where we embed the synthetic attC recombination site into the lacZ gene and measure the activity of β-galactosidase using the X-gal (5-bromo-4-chloro-3-indolyl-β-D-galactopyranoside).

### Example 5: Synthetic attC Recombination Sites Embedded into Protein-Coding Region (PKS genes EryAI, EryAII, EryAIII)

The approach for embedding synthetic attC recombination sites into protein-coding regions described above was used to generate four synthetic attC recombination sites embedded into each of the six modules of the three Saccharopolyspora erythrea genes encoding the DEBS (6-deoxyerythronolide B synthase): eryAI, eryAII, eryAIII. For each module, the region between the ketosynthase domain and the rigid part of the linker connecting the ketosynthase and the acyltransferase domain was selected. The regions are 150 bases long, and the recombination points were selected in the patches that present a certain homology among the six modules, so that upon recombination, the reconstitution of functional modules is more likely(see Target sequences of Modules 1-6 in Table 2). The algorithm described above was used to embed attC into these regions, and 3 candidate sequences were retained per module (see Modified target sequences of candidates for Modules 1-6 in Table 2). The sequences corresponding to N1-N2-N3-N4-N5-N6-N7, N8 and N9-N10-N11-N13-N14-N15-N16-N17-N18 within these modified target sequences are given for reference in Table 2.

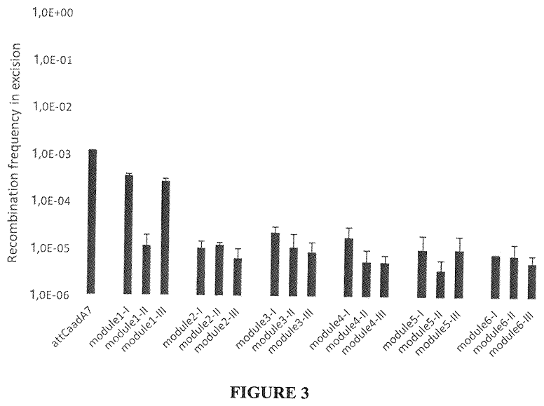

The performance of these synthetic attC recombination sites in recombination is tested, as described above, both in excision and reintegration experiments. The results of excision experiments are given in FIG. 3, where the performance of synthetic attC sites can be compared to that of a wild-type attC site, attCaadA 7.

The ability of these synthetic attC recombination sites embedded into the eryAI, eryAII, eryAIII genes to not interfere with the functionality of the resulting DEBS protein is tested, by performing HPLC purifications of the resulting secondary metabolite, as described above.

TABLE 3
sequences of the target regions for the 6 modules of DEBS-encoding genes from
Saccharopolyspora erythrea (eryAI, eryAII, eryAIII), as well as the modified
sequences of these regions corresponding to 3 candidate synthetic attC sites embedded
per module. For each modified target sequence, the sequences within it corresponding to
N1-N2-N3-N4-N5-N6-N7, N8 and N9-N10-N11-N13-N14-N15-N16-N17-N18 are given for reference.

| Module 1 | | | |
|---|---|---|---|
| Target | CTGCACGCATCGGAGCGGTCGAAGGAGATCGACTGGTCATCCGGTGCGATCAGCCTGCTCGACGA | | SEQ ID NO: 17 |
| sequence | GCCGGAGCCGTGGCCCGCCGGCGCGCGACCGCGCCGGGCGGGGGTCTCGTCGTTCGGCATCAGCG | | |
| | GCACCAACGCGCACGCCATCATC | | |
| module 1-I | Modified target sequence | CTGCACGCATCGGAGCGGTCGAAGGAGATA | SEQ ID NO: 18 |
| | | ACGTGGTCATCTGGTGCACTTAGCCTGCTC | |
| | | GACGAGCCGGAGCCGTGGCCCGCCGGCGCG | |
| | | CGACCGCGCCGGGCGGGGGTCTCGTCGTTC | |
| | | GGCATCAAAGGCACCAATGCTCATGTTATC | |
| | | TTC | |
| | N1-N2-N3-N4-N5-N6-N7 | AGATAACGTGGTCATCTGGTGCACTT | SEQ ID NO: 19 |
| | N8 | AGCCTGCTCGACGAGCCGGAGCCGTGGCCC | SEQ ID NO: 20 |
| | | GCCGGCGCGCGACCGCGCCGGGCGGGGGTC | |
| | | TCGTCGTTCGGCATCA | |
| | N9-N10-N11-N13-N14-N15-N16-N17-N18 | AAGGCACCAATGCTCATGTTATCT | SEQ ID NO: 21 |
| module 1-II | Modified target sequence | CTGCACGCATCGGAGCGGTCGAAGGCTATA | SEQ ID NO: 22 |
| | | ACTTGGTCATCTGGTGCACTTAGCCTGCTC | |
| | | GACGAGCCGGAGCCGTGGCCCGCCGGCGCG | |
| | | CGACCGCGCCGGGCGGGGGTCTCGTCGTTC | |
| | | GGCATCAAAGGCACCAATGCGCATGTTATA | |
| | | GTC | |
| | N1-N2-N3-N4-N5-N6-N7 | CTATAACTTGGTCATCTGGTGCACTT | SEQ ID NO: 23 |
| | N8 | AGCCTGCTCGACGAGCCGGAGCCGTGGCCC | SEQ ID NO: 24 |
| | | GCCGGCGCGCGACCGCGCCGGGCGGGGGTC | |
| | | TCGTCGTTCGGCATCA | |
| | N9-N10-N11-N13-N14-N15-N16-N17-N18 | AAGGCACCAATGCGCATGTTATAG | SEQ ID NO: 25 |
| module 1-III | Modified target sequence | CTGCACGCATCGGAGCGGTCGAAGGAGATC | SEQ ID NO: 26 |
| | | GACTGGAATAACGGTGCCATCTCGTACACA | |
| | | GACGAGCCGGAGCCGTGGCCCGCCGGCGCG | |
| | | CGACCGCGCCGGGCGGGGGTCTCGTCGTTC | |
| | | GGCATCACTGGTACGAATGCGCACGTTATT | |
| | | CTC | |
| | N1-N2-N3-N4-N5-N6-N7 | GAATAACGGTGCCATCTCGTACACAG | SEQ ID NO: 27 |
| | N8 | ACGAGCCGGAGCCGTGGCCCGCCGGCGCGC | SEQ ID NO: 28 |
| | | GACCGCGCCGGGCGGGGGTCTCGTCGTTCG | |
| | | GCATCA | |
| | N9-N10-N11-N13-N14-N15-N16-N17-N18 | CTGGTACGAATGCGCACGTTATTC | SEQ ID NO: 29 |
| Module 2 | | | |
| Target | TGCCGGGGCGAGAGGTCGGGCCTCATCGACTGGTCCTCCGGCGAGATCGAGCTCGCAGACGGCGT | | SEQ ID NO: 30 |
| sequence | GCGGGAGTGGTCGCCCGCCGCGGACGGGGTGCGCCGGGCAGGTGTGTCGGCGTTCGGGGTGAGCG | | |
| | GGACGAACGCGCACGTGATCATC | | |
| module 2-I | Modified target sequence | TGCCGGGGCGAGAGGTCGGGCCTGATAACG | SEQ ID NO: 31 |
| | | TGGTCGTCTGGTTCACTGGAGCTCGCAGAC | |
| | | GGCGTGCGGGAGTGGTCGCCCGCCGCGGAC | |
| | | GGGGTGCGCCGGGCAGGTGTGTCGGCGTTC | |
| | | GGGGTGACAGGAACCAACGCACACGTTATC | |
| | | ATC | |
| | N1-N2-N3-N4-N5-N6-N7 | TGATAACGTGGTCGTCTGGTTCACTG | SEQ ID NO: 32 |
| | N8 | GAGCTCGCAGACGGCGTGCGGGAGTGGTCG | SEQ ID NO: 33 |
| | | CCCGCCGCGGACGGGGTGCGCCGGGCAGGT | |
| | | GTGTCGGCGTTCGGGGTGA | |
| | N9-N10-N11-N13-N14-N15-N16-N17-N18 | CAGGAACCAACGCACACGTTATCA | SEQ ID NO: 34 |
| module 2-II | Modified target sequence | TGCCGGGGCGAGAGGTCGGGCCTCATCGAC | SEQ ID NO: 35 |
| | | TGGTCCTCCGGCGAGATAACTCTGGCGTCT | |
| | | GGTGCACGCGAGTGGTCGCCCGCCGCGGAC | |
| | | GGGGTGCGCCGGGCAGGTGTGTCGGCGTTC | |
| | | GGGGTGAGCGGCACCAACGCTCATGTTATC | |
| | | TTC | |
| | N1-N2-N3-N4-N5-N6-N7 | AGATAACTCTGGCGTCTGGTGCACGC | SEQ ID NO: 36 |
| | N8 | GAGTGGTCGCCCGCCGCGGACGGGGTGCGC | SEQ ID NO: 37 |
| | | CGGGCAGGTGTGTCGGCGTTCGGGGTGA | |
| | N9-N10-N11-N13-N14-N15-N16-N17-N18 | GCGGCACCAACGCTCATGTTATCT | SEQ ID NO: 38 |
| module 2-III | Modified target sequence | TGCCGGGGCGAGAGGTCGGGCCTCATCGAC | SEQ ID NO: 39 |
| | | TGGTCCTCCGGCGAGATCGAGCTCGCAGAC | |
| | | GGCGTGCGGGAGTGGTCGCCCGCCGCGGAC | |
| | | GGGATAACACGCAGTTCAGTACCAGCTTTC | |
| | | GGGGTGAGCGGTACTAACGCGCATGTTATC | |
| | | CTC | |
| | N1-N2-N3-N4-N5-N6-N7 | GGATAACACGCAGTTCAGTACCAGCT | SEQ ID NO: 40 |
| | N8 | TTCGGGGTG | |
| | N9-N10-N11-N13-N14-N15-N16-N17-N18 | AGCGGTACTAACGCGCATGTTATCC | SEQ ID NO: 41 |
| Module 3 | | | |
| Target | CTGCACGTCGAGGAGCCCACGCCGCACGTCGACTGGTCGTCCGGCGGCGTGGCGCTGCTGGCGGG | | SEQ ID NO: 42 |
| sequence | CAACCAGCCGTGGCGGCGCGGCGAGCGGACTCGGCGCGCCGCTGTTTCCGCGTTCGGGATCAGCG | | |
| | GGACGAATGCGCACGTGATCGTC | | |
| module 3-I | Modified target sequence | CTGCACGTCGAGGAGCCCACGCCGCACGTC | SEQ ID NO: 43 |
| | | GACTGGTCGTCCGGCGGCGTGGCGCTGCTG | |
| | | GCGGGCAACCAGCCGTGGCGGCGCGGCGAG | |
| | | CGTACAACGAGGAGTTCTGTACCAGCTTTC | |
| | | GGGATCAGCGGTACAAACGCGCACGTTGTA | |
| | | CTC | |
| | N1-N2-N3-N4-N5-N6-N7 | GTACAACGAGGAGTTCTGTACCAGCT | SEQ ID NO: 44 |
| | N8 | TTCGGGATC | |
| | N9-N10-N11-N13-N14-N15-N16-N17-N18 | AGCGGTACAAACGCGCACGTTGTAC | SEQ ID NO: 45 |
| module 3-II | Modified target sequence | CTGCACGTCGAGGAGCCCACGCCGCACGTC | SEQ ID NO: 46 |
| | | GACTGGTCGTCCGGCGGCGTGGCGCTGCTG | |
| | | GCGGGCAACCAGCCGTGGCGGCGCGGCGAG | |
| | | CGTACAACGCGGGCTGCAGTACCAGCTTTC | |
| | | GGGATCAGCGGTACTCAGGCTCACGTTGTA | |
| | | CTC | |
| | N1-N2-N3-N4-N5-N6-N7 | GTACAACGCGGGCTGCAGTACCAGCT | SEQ ID NO: 47 |
| | N8 | TTCGGGATC | |
| | N9-N10-N11-N13-N14-N15-N16-N17-N18 | AGCGGTACTCAGGCTCACGTTGTAC | SEQ ID NO: 48 |
| module 3-III | Modified target sequence | CTGCACGTCGAGGAGCCCACGCCGCACGTC | SEQ ID NO: 49 |
| | | GACTGGTCGTCCGGCGGCGTGGCGCTGCTG | |
| | | GCGGGCAACCAGCCGTGGCGGCGCGGCGAG | |
| | | CGACAACGAAGACGTCTGGTGCACGCGTTC | |
| | | GGGATCAGCGGCACCAACGCTCACGTTGTC | |
| | | GTC | |
| | N1-N2-N3-N4-N5-N6-N7 | CGACAACGAAGACGTCTGGTGCACGC | SEQ ID NO: 50 |
| | N8 | GTTCGGGATCA | SEQ ID NO: 51 |
| | N9-N10-N11-N13-N14-N15-N16-N17-N18 | GCGGCACCAACGCTCACGTTGTCG | SEQ ID NO: 52 |
| Module 4 | | | |
| Target | TTGCACGCCGACGAGCTGTCCCCGCACATCGACTGGGAGTCGGGGGCCGTGGAGGTGCTGCGCGA | | SEQ ID NO: 53 |
| sequence | GGAGGTGCCGTGGCCGGCGGGTGAGCGCCCCCGGCGGGCGGGGGTGTCGTCCTTCGGCGTCAGCG | | |
| | GAACCAACGCGCACGTGATCGTC | | |
| module 4-I | Modified target sequence | TTGCACGCCGACGAGCTGTCCCCGCACATC | SEQ ID NO: 54 |
| | | GACTGGGAGTCGGGACAACTGGAAGTTCTG | |
| | | CGCCAGGAGGTGCCGTGGCCGGCGGGTGAG | |
| | | CGCCCCCGGCGGGCGGGGGTGTCGTCCTTC | |
| | | GGCGTCTCCGGCGCAAACTCACATGTTGTC | |
| | | CTC | |
| | N1-N2-N3-N4-N5-N6-N7 | GGACAACTGGAAGTTCTGCGCCAGGA | SEQ ID NO: 55 |
| | N8 | GGTGCCGTGGCCGGCGGGTGAGCGCCCCCG | SEQ ID NO: 56 |
| | | GCGGGCGGGGGTGTCGTCCTTCGGCGTC | |
| | N9-N10-N11-N13-N14-N15-N16-N17-N18 | TCCGGCGCAAACTCACATGTTGTCC | SEQ ID NO: 57 |
| module 4-II | Modified target sequence | TTGCACGCCGACGAGCTGTCCCCGCACATC | SEQ ID NO: 58 |
| | | GACTGGGAGTCGGGGGCCGTGGAGGTGCTG | |
| | | CGCGAGGAGGTGCCGTGGCCGGCGGGTGAG | |
| | | CGCCCCCACAACGCAGGCGTCTCGTCCACC | |
| | | GGCGTCACGGGGACGAACGCTCACGTTGTG | |
| | | GTC | |
| | N1-N2-N3-N4-N5-N6-N7 | CCACAACGCAGGCGTCTCGTCCACCG | SEQ ID NO: 59 |
| | N8 | GCGTCA | |
| | N9-N10-N11-N13-N14-N15-N16-N17-N18 | CGGGGACGAACGCTCACGTTGTGG | SEQ ID NO: 60 |
| module 4-III | Modified target sequence | TTGCACGCCGACGAGCTGTCCCCGCACATC | SEQ ID NO: 61 |
| | | GACTGGGAGTCGGGGGCCGTGGAGGTGCTG | |
| | | CGCGAGGAGGTGCCGTGGCCGGCGGGTGAG | |
| | | CGCCCTAACCGCGCAGGCGTTTCCAGCTTC | |
| | | GGCGTCAGCGGAAACCCTGCGCATGTTAGG | |
| | | GTC | |
| | N1-N2-N3-N4-N5-N6-N7 | CCCTAACCGCGCAGGCGTTTCCAGCT | SEQ ID NO: 62 |
| | N8 | TCGGCGTC | |
| | N9-N10-N11-N13-N14-N15-N16-N17-N18 | AGCGGAAACCCTGCGCATGTTAGGG | SEQ ID NO: 63 |
| Module 5 | | | |
| Target | CTGCACTTCGACGAGCCCTCGCCGCAGATCGAGTGGGACCTGGGCGCGGTGTCGGTGGTGTCGCA | | SEQ ID NO: 64 |
| sequence | GGCGCGGTCGTGGCCCGCCGGCGAGAGGCCCCGCAGGGCGGGCGTCTCCTCGTTCGGCATCAGCG | | |
| | GCACCAACGCGCACGTCATCGTC | | |
| module 5-I | Modified target sequence | CTGCACTTCGACGAGCCCTCGCCGCAGATC | SEQ ID NO: 65 |
| | | GAGTGGGACCTGGGCGCGATAACAGTTGCG | |
| | | TCTCGTGCACGCTCGTGGCCCGCCGGCGAG | |
| | | AGGCCCCGCAGGGCGGGCGTCTCCTCGTTC | |
| | | GGCATCAGCGGCACGAACGCTCATGTTATC | |
| | | GTC | |
| | N1-N2-N3-N4-N5-N6-N7 | CGATAACAGTTGCGTCTCGTGCACGC | SEQ ID NO: 66 |
| | N8 | TCGTGGCCCGCCGGCGAGAGGCCCCGCAGG | SEQ ID NO: 67 |
| | | GCGGGCGTCTCCTCGTTCGGCATCA | |
| | N9-N10-N11-N13-N14-N15-N16-N17-N18 | GCGGCACGAACGCTCATGTTATCG | SEQ ID NO: 68 |
| module 5-II | Modified target sequence | CTGCACTTCGACGAGCCCTCGCCGCAGATC | SEQ ID NO: 69 |
| | | GAGTGGGACCTGGGCGCGATAACTGTAGTG | |
| | | TCTCGTGCACGCTCGTGGCCCGCCGGCGAG | |
| | | AGGCCCCGCAGGGCGGGCGTCTCCTCGTTC | |
| | | GGCATCAGCGGCACGAACACTCATGTTATC | |
| | | GTC | |
| | N1-N2-N3-N4-N5-N6-N7 | CGATAACTGTAGTGTCTCGTGCACGC | SEQ ID NO: 70 |
| | N8 | TCGTGGCCCGCCGGCGAGAGGCCCCGCAGG | SEQ ID NO: 71 |
| | | GCGGGCGTCTCCTCGTTCGGCATCA | |
| | N9-N10-N11-N13-N14-N15-N16-N17-N18 | GCGGCACGAACACTCATGTTATCG | SEQ ID NO: 72 |
| module 5-III | Modified target sequence | CTGCACTTCGACGAGCCCTCGCCGCAGATC | SEQ ID NO: 73 |
| | | GAGTGGGACCTGGGCGCGATAACGGTAGTG | |
| | | TCTCGTGCACGCTCGTGGCCCGCCGGCGAG | |
| | | AGGCCCCGCAGGGCGGGCGTCTCCTCGTTC | |
| | | GGCATCAGCGGCACGAACATTCATGTTATC | |
| | | GTC | |
| | N1-N2-N3-N4-N5-N6-N7 | CGATAACGGTAGTGTCTCGTGCACGC | SEQ ID NO: 74 |
| | N8 | TCGTGGCCCGCCGGCGAGAGGCCCCGCAGG | SEQ ID NO: 75 |
| | | GCGGGCGTCTCCTCGTTCGGCATCA | |
| | N9-N10-N11-N13-N14-N15-N16-N17-N18 | GCGGCACGAACATTCATGTTATCG | SEQ ID NO: 76 |
| Module 6 | | | |
| Target | TGCCGCGGCGAGCGGTCGCCGCTGATCGAATGGTCCTCGGGTGGTGTGGAACTTGCCGAGGCCGT | | SEQ ID NO: 77 |
| sequence | GAGCCCGTGGCCTCCGGCCGCGGACGGGGTGCGCCGGGCCGGTGTGTCGGCGTTCGGGGTGAGCG | | |
| | GGACGAACGCGCACGTGATCATC | | |
| module 6-I | Modified target sequence | TGCCGCGGAGAACGTAGTCCTCTCGTGCAC | SEQ ID NO: 78 |
| | | TGGTCCTCGGGTGGTGTGGAACTTGCCGAG | |
| | | GCCGTGAGCCCGTGGCCTCCGGCCGCGGAC | |
| | | GGGGTGCGCCGGGCCGGTGTGTCGGCGTTC | |
| | | GGGGTGACAGGCACGAAGGCACACGTTCTC | |
| | | CTC | |
| | N1-N2-N3-N4-N5-N6-N7 | GGAGAACGTAGTCCTCTCGTGCACTG | SEQ ID NO: 79 |
| | N8 | GTCCTCGGGTGGTGTGGAACTTGCCGAGGC | SEQ ID NO: 80 |
| | | CGTGAGCCCGTGGCCTCCGGCCGCGGACGG | |
| | | GGTGCGCCGGGCCGGTGTGTCGGCGTTCGG | |
| | | GGTGA | |
| | N9-N10-N11-N13-N14-N15-N16-N17-N18 | CAGGCACGAAGGCACACGTTCTCC | SEQ ID NO: 81 |
| module 6-II | Modified target sequence | TGCCGCGGCGAGCGGTCGCCGCTGATAACT | SEQ ID NO: 82 |
| | | TGGTCATCTGGTGGAGTGGAACTTGCCGAG | |
| | | GCCGTGAGCCCGTGGCCTCCGGCCGCGGAC | |
| | | GGGGTGCGCCGGGCCGGTGTGTCGGCGTTC | |
| | | GGGGTGACACCCACCAATGCTCATGTTATC | |
| | | ATC | |
| | N1-N2-N3-N4-N5-N6-N7 | TGATAACTTGGTCATCTGGTGGAGTG | SEQ ID NO: 83 |
| | N8 | GAACTTGCCGAGGCCGTGAGCCCGTGGCCT | SEQ ID NO: 84 |
| | | CCGGCCGCGGACGGGGTGCGCCGGGCCGGT | |
| | | GTGTCGGCGTTCGGGGTGA | |
| | N9-N10-N11-N13-N14-N15-N16-N17-N18 | CACCCACCAATGCTCATGTTATCA | SEQ ID NO: 85 |
| module 6-III | Modified target sequence | TGCCGCGGCGAGCGGTCGCCGCTGATCGAA | SEQ ID NO: 86 |
| | | TGGGATAACGGTGGCGTCTCGTGCACGCAG | |
| | | GCCGTGAGCCCGTGGCCTCCGGCCGCGGAC | |
| | | GGGGTGCGCCGGGCCGGTGTGTCGGCGTTC | |
| | | GGGGTGAGCGGCACGAACGCCCATGTTATC | |
| | | CTC | |
| | N1-N2-N3-N4-N5-N6-N7 | GGATAACGGTGGCGTCTCGTGCACGC | SEQ ID NO: 87 |
| | N8 | AGGCCGTGAGCCCGTGGCCTCCGGCCGCGG | SEQ ID NO: 88 |
| | | ACGGGGTGCGCCGGGCCGGTGTGTCGGCGT | |
| | | TCGGGGTGA | |
| | N9-N10-N11-N13-N14-N15-N16-N17-N18 | GCGGCACGAACGCCCATGTTATCC | SEQ ID NO: 89 |

### REFERENCES

1. Escudero, J. A., Loot, C., Nivina, A. & Mazel, D. The Integron: Adaptation On Demand. Microbiol Spectr 3, MDNA3-0019-2014 (2015).

2. Collis, C. M., Grammaticopoulos, G., Briton, J., Stokes, H. W. & Hall, R. M. Site-specific insertion of gene cassettes into integrons. Mol. Microbiol. 9, 41-52 (1993).

3. Cambray, G., Guerout, A.-M. & Mazel, D. Integrons. Annu. Rev. Genet. 44, 141-166 (2010).

4. Mazel, D. Integrons: agents of bacterial evolution. Nat. Rev. Microbiol. 4, 608-620 (2006).

5. Partridge, S. R., Tsafnat, G., Coiera, E. & Iredell, J. R. Gene cassettes and cassette arrays in mobile resistance integrons. FEMS Microbiol. Rev. 33, 757-784 (2009).

6. Francia, M. V., Zabala, J. C., la Cruz, de, F. & Garcia Lobo, J. M. The IntI1 integron integrase preferentially binds single-stranded DNA of the attC site. Journal of Bacteriology 181, 6844-6849 (1999).

7. Johansson, C., Kamali-Moghaddam, M. & Sundström, L. Integron integrase binds to bulged hairpin DNA. Nucleic Acids Research 32, 4033-4043 (2004).

8. Bouvier, M., Demarre, G. & Mazel, D. Integron cassette insertion: a recombination process involving a folded single strand substrate. EMBO J. 24, 4356-4367 (2005).

9. Bouvier, M., Ducos-Galand, M., Loot, C., Bikard, D. & Mazel, D. Structural features of single-stranded integron cassette attC sites and their role in strand selection. PLoS Genet 5, e1000632 (2009).

10. MacDonald, D., Demarre, G., Bouvier, M., Mazel, D. & Gopaul, D. N. Structural basis for broad DNA-specificity in integron recombination. Nature 440, 1157-1162 (2006).

11. Frumerie, C., Ducos-Galand, M., Gopaul, D. N. & Mazel, D. The relaxed requirements of the integron cleavage site allow predictable changes in integron target specificity. Nucleic Acids Research 38, 559-569 (2010).

12. Escudero, J. A. et al. Unmasking the ancestral activity of integron integrases reveals a smooth evolutionary transition during functional innovation. Nature Communications 6, 1-12 (2016).

13. Bikard, D., Julie-Galau, S., Cambray, G. & Mazel, D. The synthetic integron: an in vivo genetic shuffling device. Nucleic Acids Research 38, e153-e153 (2010).

14. Hertweck, C. The biosynthetic logic of polyketide diversity. Angew. Chem. Int. Ed. Engl. 48, 4688-4716 (2009).

15. Menzella, H. G. et al. Combinatorial polyketide biosynthesis by de novo design and rearrangement of modular polyketide synthase genes. Nature Biotechnology 23, 1171-1176 (2005).

16. Menzella, H. G., Carney, J. R. & Santi, D. V. Rational design and assembly of synthetic trimodular polyketide synthases. Chem. Biol. 14, 143-151 (2007).

17. Lorenz, R. et al. ViennaRNA Package 2.0. Algorithms Mol Biol 6, 26 (2011).

18. Nivina, A. et al. Efficiency of integron cassette insertion in correct orientation is ensured by the interplay of the three unpaired features of attC recombination sites, Nucleic Acids Res.; 44 (16):7792-803 (2016).

19. Zuker, M. Mfold web server for nucleic acid folding and hybridization prediction. Nucleic Acids Research 31, 3406-3415 (2003).

20. Demarre, G. et al. A new family of mobilizable suicide plasmids based on broad host range R388 plasmid (IncW) and RP4 plasmid (IncPalpha) conjugative machineries and their cognate Escherichia coli host strains. Research in Microbiology 156, 245-255 (2005).

1. A recombinant nucleic acid comprising a protein coding sequence of a multidomain protein, wherein the protein coding sequence comprises at least one synthetic attC recombination site.

2. The recombinant nucleic acid of claim 1, wherein said at least one synthetic attC recombination site has a sequence SEQ ID NO: 1 of formula
N1-N2-N3-N4-N5-N6-N7-N8-N9-N10-N11-N12-N13-N14-N15-N16-N17-N18 wherein:
N1 is 0-10 nt long;
N2 is 4 nt long and at least the last 3 nt of N2 are reverse-complementary to the first 3 nt of N17,
N3 is 5-8 nt long and it is not reverse-complementary to N16, even though upon the formation of the intramolecular imperfect hairpin, some pairings between the bases of N3 and N16 are possible;
N4 is 2-4 nt long;
N5 is 2-4 nt long;
N6 is 2-4 nt long;
N7 is from 3 nt to 30 nt long;
N8 is from 3 nt to 100 nt long;
N9 is from 3 nt to 30 nt long;
N10 is present or absent, if present then it is one of the “Extrahelical bases”;
N11 is 2-4 nt long;
N12 can be present or absent, if present then it is one of the “Extrahelical bases”;
N13 is from 2 to 4 nt long;
N14 is present or absent; if present, then it is one of the “Extrahelical bases”;
N15 is from 2 to 4 nt long;
N16 is preferentially from 5 to 8 nt long, it is not reverse-complementary to N3, even though upon the formation of the intramolecular imperfect hairpin, some pairings between the bases of N3 and N16 are possible;
N17 is 4 nt long, and at least the first 3 nt of N17 are reverse-complementary to the last 3 nt of N2; and
N18 is 0-10 nt long.

3. The recombinant nucleic acid of claim 1, wherein said at least one synthetic attC recombination site a sequence chosen from the group consisting of SEQ ID NO: 3 to SEQ ID NO: 16 and SEQ ID NO: 90 to SEQ ID NO: 107.

4-10. (canceled)

11. A recombinant cell comprising a recombinant nucleic acid according to claim 3.

12. A library comprising a plurality of different recombinant nucleic acids according to claim 3.

13. A library comprising a plurality of different recombinant cells according to claim 11.

14. A method of making a recombinant nucleic acid encoding a recombinant multidomain protein, comprising:
providing a first recombinant nucleic acid comprising a protein coding sequence of a multidomain protein, wherein the protein coding sequence comprises at least one synthetic attC recombination site;
providing a second recombinant nucleic acid comprising a protein coding sequence of a multidomain protein, wherein the protein coding sequence comprises at least one synthetic attC recombination site; and
contacting the first and second recombinant nucleic acids with an integrase protein to thereby induce recombination between the at least one synthetic attC recombination site present in the first recombinant nucleic acid and the at least one synthetic attC recombination site present in the second recombinant nucleic acid, to thereby provide a recombined recombinant nucleic acid that encodes the recombinant multidomain protein.

15. The method of claim 14, wherein, wherein said at least one synthetic attC recombination site has a sequence SEQ ID NO: 1 of formula
N1-N2-N3-N4-N5-N6-N7-N8-N9-N10-N11-N12-N13-N14-N15-N16-N17-N18 wherein:
N1 is 0-10 nt long;
N2 is 4 nt long and at least the last 3 nt of N2 are reverse-complementary to the first 3 nt of N17,
N3 is 5-8 nt long and it is not reverse-complementary to N16, even though upon the formation of the intramolecular imperfect hairpin, some pairings between the bases of N3 and N16 are possible;
N4 is 2-4 nt long;
N5 is 2-4 nt long;
N6 is 2-4 nt long;
N7 is from 3 nt to 30 nt long;
N8 is from 3 nt to 100 nt long;
N9 is from 3 nt to 30 nt long;
N10 is present or absent, if present then it is one of the “Extrahelical bases”;
N11 is 2-4 nt long;
N12 can be present or absent, if present then it is one of the “Extrahelical bases”;
N13 is from 2 to 4 nt long;
N14 is present or absent; if present, then it is one of the “Extrahelical bases”;
N15 is from 2 to 4 nt long;
N16 is preferentially from 5 to 8 nt long, it is not reverse-complementary to N3, even though upon the formation of the intramolecular imperfect hairpin, some pairings between the bases of N3 and N16 are possible;
N17 is 4 nt long, and at least the first 3 nt of N17 are reverse-complementary to the last 3 nt of N2; and
N18 is 0-10 nt long.

16. The method of claim 14, wherein said at least one synthetic attC recombination site a sequence chosen from the group consisting of SEQ ID NO: 3 to SEQ ID NO: 16 and SEQ ID NO: 90 to SEQ ID NO: 107.

17-18. (canceled)

19. A method of making a recombinant nucleic acid encoding a recombinant multidomain protein, comprising:
providing a recombinant nucleic acid comprising a plurality of protein coding sequences of multidomain proteins, wherein each of the protein coding sequences comprises a synthetic attC recombination site; and
contacting the recombinant nucleic acid with an integrase protein to thereby induce recombination between at least one pair of the synthetic attC recombination sites, to thereby provide a recombined recombinant nucleic acid that encodes the recombinant multidomain protein.

20. The method of claim 19, wherein, wherein said synthetic attC recombination site has a sequence SEQ ID NO: 1 of formula
N1-N2-N3-N4-N5-N6-N7-N8-N9-N10-N11-N12-N13-N14-N15-N16-N17-N18 wherein:
N1 is 0-10 nt long;
N2 is 4 nt long and at least the last 3 nt of N2 are reverse-complementary to the first 3 nt of N17,
N3 is 5-8 nt long and it is not reverse-complementary to N16, even though upon the formation of the intramolecular imperfect hairpin, some pairings between the bases of N3 and N16 are possible;
N4 is 2-4 nt long;
N5 is 2-4 nt long;
N6 is 2-4 nt long;
N7 is from 3 nt to 30 nt long;
N8 is from 3 nt to 100 nt long;
N9 is from 3 nt to 30 nt long;
N10 is present or absent, if present then it is one of the “Extrahelical bases”;
N11 is 2-4 nt long;
N12 can be present or absent, if present then it is one of the “Extrahelical bases”;
N13 is from 2 to 4 nt long;
N14 is present or absent; if present, then it is one of the “Extrahelical bases”;
N15 is from 2 to 4 nt long;
N16 is preferentially from 5 to 8 nt long, it is not reverse-complementary to N3, even though upon the formation of the intramolecular imperfect hairpin, some pairings between the bases of N3 and N16 are possible;
N17 is 4 nt long, and at least the first 3 nt of N17 are reverse-complementary to the last 3 nt of N2; and
N18 is 0-10 nt long.

21. The method of claim 19, wherein said synthetic attC recombination site a sequence chosen from the group consisting of SEQ ID NO: 3 to SEQ ID NO: 16 and SEQ ID NO: 90 to SEQ ID NO: 107.

22-23. (canceled)

24. The method of claim 21, wherein contacting the recombinant nucleic acid(s) with integrase protein is by a process comprising introducing a recombinant nucleic acid encoding the integrase protein into a cell comprising the recombinant nucleic acid(s) and expressing the integrase protein.

25. The method of claim 21, wherein the recombinant multidomain protein is a recombinant PKS.

26. The method of claim 21, wherein the recombinant multidomain protein is a recombinant NRPS.

27. A method of making a recombinant multidomain protein, comprising making a recombinant nucleic acid encoding a multidomain protein by the method of claim 14 and expressing the recombinant multidomain protein.

28. A recombinant cell comprising a recombinant nucleic acid made by the method of claim 14.

29-30. (canceled)

31. A library comprising a plurality of different recombinant nucleic acids made by the method of claim 14.

32. A library comprising a plurality of different recombinant cells according to claim 28.

33. A library comprising a plurality of different recombinant multidomain proteins according to claim 27.
